SEQUENCE LISTING (69 sequences)

SEQ ID NO: 1 - protein, 43 aa, Artificial Sequence
  Synthetic peptide
DAEFRHDSGYEVHHQKLVFFAEDVGSNKGAIIGLMVGGVVIAT

SEQ ID NO: 2 - protein, 43 aa, Artificial Sequence
  Synthetic peptide
DAEFDHRSGYEVHSQKLVFFAEDVGSNKGAIIGLMVGGVVIAT

SEQ ID NO: 3 - protein, 43 aa, Artificial Sequence
  Synthetic peptide
DAEFRDHSGYHVEHQKLVFFAEDVGSNKGAIIGLMVGGVVIAT

SEQ ID NO: 4 - protein, 43 aa, Artificial Sequence
  Synthetic peptide
DAEFRHDSGYEVHHQKLVFFAEDVGSNKGAIIGLMVGGVVIAA

SEQ ID NO: 5 - protein, 43 aa, Artificial Sequence
  Synthetic peptide
DAEFRHDSGYEVHHQKLVFFAEDVGSNKGAIIGLMVGGVVIAV

SEQ ID NO: 6 - protein, 43 aa, Artificial Sequence
  Synthetic peptide
DAEFRHDSGYEVHHQKLVFFAEDVGSNKGAIIGLMVGGVVIAL

SEQ ID NO: 7 - protein, 43 aa, Artificial Sequence
  Synthetic peptide
DAEFRHDSGYEVHHQKLVFFAEDVGSNKGAIIGLMVGGVVIAI

SEQ ID NO: 8 - protein, 43 aa, Artificial Sequence
  Synthetic peptide
  MOD_RES (35)..(35): Nle
  MOD_RES (37)..(37): Xaa denotes D-proline
DAEFRHDSGYEVHHQKLVFFAEDVGSNKGAIIGLXVXGVVIAT

SEQ ID NO: 9 - protein, 43 aa, Artificial Sequence
  Synthetic peptide
  MOD_RES (1)..(1): Xaa denotes biotinyl-L-lysine
XAEFRHDSGYEVHHQKLVFFAEDVGSNKGAIIGLMVGGVVIAT

SEQ ID NO: 10 - protein, 43 aa, Artificial Sequence
  Synthetic peptide
  MOD_RES (37)..(37): Xaa denotes D-proline
DAEFRHDSGYEVDEQKLVFFAEDVGSNKGAIIGLMVXGVVIST

SEQ ID NO: 11 - protein, 43 aa, Artificial Sequence
  Synthetic peptide
  MOD_RES (37)..(37): Xaa denotes D-proline
DAEFRHDSGYEVDEQKLVFFAEDVGSNKGAIIGLVVXGVVIST

SEQ ID NO: 12 - protein, 33 aa, Artificial Sequence
  Synthetic peptide
  MOD_RES (27)..(27): Xaa denotes D-proline
EVDEQKLVFFAEDVGSNKGAIIGLMVXGVVIST

SEQ ID NO: 13 - protein, 33 aa, Artificial Sequence
  Synthetic peptide
  MOD_RES (27)..(27): Xaa denotes D-proline
EVDEQKLVFFAEDVGSNKGAIIGLVVXGVVIST

SEQ ID NO: 14 - protein, 37 aa, Artificial Sequence
  Synthetic peptide
DSGYEVQQQKLVFFAEDVGSNKGAIIGLMVGGVVIAV

SEQ ID NO: 15 - protein, 37 aa, Artificial Sequence
  Synthetic peptide
DSGYEVQQQQLVFFAEDVGSNKGAIIGLMVGGVVIAV

SEQ ID NO: 16 - protein, 37 aa, Artificial Sequence
  Synthetic peptide
  MOD_RES (31)..(31): Xaa denotes D-proline
DSGFEVQQQKLVFFAEDVGSNKGAIIGLMVXGVVIAV

SEQ ID NO: 17 - protein, 37 aa, Artificial Sequence
  Synthetic peptide
  MOD_RES (1)..(1): Orn
  MOD_RES (5)..(5): Orn
  MOD_RES (29)..(29): Nle
XSGYXVDDQELVFFAEDVGSNKGAIIGLXVGGVVIAT

SEQ ID NO: 18 - protein, 27 aa, Artificial Sequence
  Synthetic peptide
  MOD_RES (21)..(21): Xaa denotes D-proline
LVFFAEDVGSNKGAIIGLMVXGVVIST

SEQ ID NO: 19 - protein, 27 aa, Artificial Sequence
  Synthetic peptide
  MOD_RES (21)..(21): Xaa denotes D-proline
LVFFAEDVGSNKGAIIGLVVXGVVIST

SEQ ID NO: 20 - protein, 21 aa, Artificial Sequence
  Synthetic peptide
  MOD_RES (1)..(1): ACETYLATION
  MOD_RES (15)..(15): Xaa denotes D-proline
DVGSNKGAIIGLMVXGVVIST

SEQ ID NO: 21 - protein, 21 aa, Artificial Sequence
  Synthetic peptide
  MOD_RES (1)..(1): ACETYLATION
  MOD_RES (15)..(15): Xaa denotes D-proline
DVGSNKGAIIGLVVXGVVIST

SEQ ID NO: 22 - protein, 16 aa, Artificial Sequence
  Synthetic peptide
  MOD_RES (10)..(10): Xaa denotes D-proline
KGAIIGLMVXGVVIST

SEQ ID NO: 23 - protein, 16 aa, Artificial Sequence
  Synthetic peptide
  MOD_RES (10)..(10): Xaa denotes D-proline
KGAIIGLVVXGVVIST

SEQ ID NO: 24 - protein, 42 aa, Artificial Sequence [sequence omitted]

SEQ ID NO: 25 - protein, 42 aa, Artificial Sequence [sequence omitted]

SEQ ID NO: 26 - protein, 42 aa, Artificial Sequence
  Synthetic peptide
DAEFRGDSGYEVHHQKLVFFAEDVGSNKGAIIGLMVGGVVIA

SEQ ID NO: 27 - protein, 42 aa, Artificial Sequence
  Synthetic peptide
  MOD_RES (1)..(1): Amino acid labeled with biotin
  MOD_RES (10)..(10): Xaa denotes p-benzoyl-L-phenylalanine
DAEFRHDSGXEVHHQKLVFFAEDVGSNKGAIIGLMVGGVVIA

SEQ ID NO: 28 - protein, 42 aa, Artificial Sequence
  Synthetic peptide
DAEFRHDSGYEVHHQKLVFFAGDVGSNKGAIIGLMVGGVVIA

SEQ ID NO: 29 - protein, 42 aa, Artificial Sequence
  Synthetic peptide
DAEFRHDSGYEVHHQKLVFFAEDVGANKGAIIGLMVGGVVIA

SEQ ID NO: 30 - protein, 42 aa, Artificial Sequence
  Synthetic peptide
DAEFRHDSGYEVHHQKLVFFAEDVGSAKGAIIGLMVGGVVIA

SEQ ID NO: 31 - protein, 42 aa, Artificial Sequence
  Synthetic peptide
DAEFDHRSGYEVHSQKLVFFAEDVGSNKGAIIGLMVGGVVIA

SEQ ID NO: 32 - protein, 42 aa, Artificial Sequence
  Synthetic peptide
DAEFDHRSGYEVHSQKLVFFAGDVGSNKGAIIGLMVGGVVIA

SEQ ID NO: 33 - protein, 42 aa, Artificial Sequence
  Synthetic peptide
DAEFDHRSGFEVHSQKLVFFAGDVGSNKGAIIGLMVGGVVIA

SEQ ID NO: 34 - protein, 42 aa, Artificial Sequence
  Synthetic peptide
  MOD_RES (1)..(1): Amino acid labeled with biotin
  MOD_RES (35)..(35): Nle
  MOD_RES (37)..(37): Xaa denotes D-proline
DAEFDHRSGYEVHSQKLVFFAEDVGSNKGAIIGLXVXGVVIA

SEQ ID NO: 35 - protein, 42 aa, Artificial Sequence
  Synthetic peptide
DAEFRDHSGYEVHHQKLVFFAEDVGSNKGAIIGLMVGGVVIA

SEQ ID NO: 36 - protein, 42 aa, Artificial Sequence
  Synthetic peptide
DAEFRDHSGYEVHHQKLVFFAGDVGSNKGAIIGLMVGGVVIA

SEQ ID NO: 37 - protein, 42 aa, Artificial Sequence
  Synthetic peptide
DAEFRDHSGYHVEHQKLVFFAEDVGSNKGAIIGLMVGGVVIA

SEQ ID NO: 38 - protein, 42 aa, Artificial Sequence
  Synthetic peptide
DAEFRDHSGFHVEHQKLVFFAEDVGSNKGAIIGLMVGGVVIA

SEQ ID NO: 39 - protein, 42 aa, Artificial Sequence
  Synthetic peptide
DAEFRDHSGYHVEHQKLVFFAGDVGSNKGAIIGLMVGGVVIA

SEQ ID NO: 40 - protein, 42 aa, Artificial Sequence
  Synthetic peptide
  MOD_RES (37)..(37): Xaa denotes D-proline
DAEFRHDSGYEVHHQKLVFFAEDVGSNKGAIIGLMVXGVVIA

SEQ ID NO: 41 - protein, 42 aa, Artificial Sequence
  Synthetic peptide
  MOD_RES (35)..(35): Nle
  MOD_RES (37)..(37): Xaa denotes D-proline
DAEFRHDSGYEVHHQKLVFFAEDVGSNKGAIIGLXVXGVVIA

SEQ ID NO: 42 - protein, 42 aa, Artificial Sequence
  Synthetic peptide
  MOD_RES (35)..(35): Nle
  MOD_RES (37)..(37): Xaa denotes D-proline
DAEFRDHSGYHVEHQKLVFFAEDVGSNKGAIIGLXVXGVVIA

SEQ ID NO: 43 - protein, 42 aa, Artificial Sequence; identical to SEQ ID NO: 41
  Synthetic peptide
  MOD_RES (1)..(1): Amino acid labeled with biotin
  MOD_RES (35)..(35): Nle
  MOD_RES (37)..(37): Xaa denotes D-proline

SEQ ID NO: 44 - protein, 42 aa, Artificial Sequence
  Synthetic peptide
  MOD_RES (1)..(1): Amino acid labeled with biotin
  MOD_RES (10)..(10): Xaa denotes p-benzoyl-L-phenylalanine
  MOD_RES (37)..(37): Xaa denotes D-proline
DAEFRHDSGXEVHHQKLVFFAEDVGSNKGAIIGLMVXGVVIA

SEQ ID NO: 45 - protein, 42 aa, Artificial Sequence
  Synthetic peptide
  MOD_RES (35)..(35): Nle
DCEFRHDSGKEVHHQKLVFFAEDVGSNKGAIIGLXVGGVVIA

SEQ ID NO: 46 - protein, 42 aa, Artificial Sequence
  Synthetic peptide
  MOD_RES (1)..(1): Xaa denotes biotinyl-L-lysine
XAEFRHDSGYEVHHQKLVFFAEDVGSNKGAIIGLMVGGVVIA

SEQ ID NO: 47 - protein, 41 aa, Artificial Sequence
  Synthetic peptide
  MOD_RES (1)..(1): Orn
  MOD_RES (3)..(3): Orn
  MOD_RES (7)..(7): Orn
  misc_feature (10)..(10): Xaa can be any naturally occurring amino acid
  MOD_RES (11)..(11): Orn
  misc_feature (34)..(34): Xaa can be any naturally occurring amino acid
  MOD_RES (35)..(35): Nle
XAXFEDSGYXVDDQELVFFAEDVGSNKGAIIGLXVGGVVIA

SEQ ID NO: 48 - protein, 42 aa, Artificial Sequence
  Synthetic peptide
  MOD_RES (1)..(1): Orn
  MOD_RES (1)..(1): Amino acid labeled with biotin
  MOD_RES (3)..(3): Orn
  MOD_RES (7)..(7): Orn
  MOD_RES (11)..(11): Orn
  MOD_RES (35)..(35): Nle
XAXFEDXSGYXVDDQELVFFAEDVGSNKGAIIGLXVGGVVIA

SEQ ID NO: 49 - protein, 41 aa, Artificial Sequence
  Synthetic peptide
  MOD_RES (36)..(36): Xaa denotes D-proline
AEFRHDSGYEVHHQKLVFFAGDVGSNKGAIIGLMVXGVVIA

SEQ ID NO: 50 - protein, 36 aa, Artificial Sequence
  Synthetic peptide
DSGYEVQQQKLVFFAEDVGSNKGAIIGLMVGGVVIA

SEQ ID NO: 51 - protein, 36 aa, Artificial Sequence
  Synthetic peptide
  MOD_RES (1)..(1): Orn
  MOD_RES (5)..(5): Orn
  MOD_RES (29)..(29): Nle
XSGYXVDDQELVFFAEDVGSNKGAIIGLXVGGVVIA

SEQ ID NO: 52 - protein, 36 aa, Artificial Sequence
  Synthetic peptide
  MOD_RES (1)..(1): Orn
  MOD_RES (5)..(5): Orn
  MOD_RES (29)..(29): Nle
XSGFXVDDQELVFFAEDVGSNKGAIIGLXVGGVVIA

SEQ ID NO: 53 - protein, 31 aa, Artificial Sequence
  Synthetic peptide
  MOD_RES (24)..(24): Nle
VDDQGLVFFAEDVGSNKGAIIGLXVGGVVIA

SEQ ID NO: 54 - protein, 31 aa, Artificial Sequence
  Synthetic peptide
  MOD_RES (1)..(1): Amino acid labeled with biotin
  MOD_RES (24)..(24): Nle
VDDQELVFFAEDVGSNKGAIIGLXVGGVVIA

SEQ ID NO: 55 - protein, 22 aa, Artificial Sequence
  Synthetic peptide
  MOD_RES (1)..(1): Amino acid labeled with biotin
  MOD_RES (2)..(2): Orn
  MOD_RES (4)..(4): Orn
DXDXGKGAIIGLMVGGVVIAKK

SEQ ID NO: 56 - protein, 26 aa, Artificial Sequence
  Synthetic peptide
LVFFAEDVGSNKGAIIGLMVGGVVIA

SEQ ID NO: 57 - protein, 26 aa, Artificial Sequence
  Synthetic peptide
  MOD_RES (19)..(19): Nle
LVFFAEDVGSNKGAIIGLXVGGVVIA

SEQ ID NO: 58 - protein, 20 aa, Artificial Sequence
  Synthetic peptide
  MOD_RES (1)..(1): Amino acid labeled with biotin
KKKKGKGAIIGLMVGGVVIA

SEQ ID NO: 59 - protein, 40 aa, Artificial Sequence
  Synthetic peptide
  MOD_RES (1)..(1): Amino acid labeled with fluoroscein
DAEFRHDSGYEVHHQKLVFFAEDVGSNKGAIIGLMVGGVV

SEQ ID NO: 60 - protein, 40 aa, Artificial Sequence
  Synthetic peptide
  misc_feature (1)..(1): Xaa can be any naturally occurring amino acid
XAEFRHDSGYEVHHQKLVFFAEDVGSNKGAIIGLMVGGVV

SEQ ID NO: 61 - protein, 40 aa, Artificial Sequence
  Synthetic peptide
  MOD_RES (1)..(1): Amino acid labeled with biotin
  MOD_RES (10)..(10): Xaa denotes p-benzoyl-L-phenylalanine
DAEFRHDSGXEVHHQKLVFFAEDVGSNKGAIIGLMVGGVV

SEQ ID NO: 62 - protein, 40 aa, Artificial Sequence
  Synthetic peptide
  MOD_RES (35)..(35): Nle
DCEFRHDSGKEVHHQKLVFFAEDVGSNKGAIIGLXVGGVV

SEQ ID NO: 63 - protein, 24 aa, Artificial Sequence
  Synthetic peptide
  MOD_RES (1)..(1): Amino acid labeled with biotin
LVFFAEDVGSNKGAIIGLMVGGVV

SEQ ID NO: 64 - protein, 9 aa, Artificial Sequence
  Synthetic peptide
  MISC_FEATURE (1)..(1): Xaa denotes a peptide of 25 to 34 amino acid residues in length
  MISC_FEATURE (2)..(5): Xaa denotes a natural or modified hydrophobic amino acid residue
  MISC_FEATURE (6)..(7): Xaa denotes a glycyl-glycyl, glycyl-prolyl, or prolyl-glycyl dipeptide,
  MISC_FEATURE (8)..(8): Xaa denotes a natural or modified hydrophobic amino acid residue
  MISC_FEATURE (9)..(9): Xaa denotes 1 to 3 natural or modified hydrophobic amino acid residues
XXXXXXXXX

SEQ ID NO: 65 - protein, 15 aa, Artificial Sequence
  Synthetic peptide
  MISC_FEATURE (1)..(1): Xaa denotes a peptide of 0 to 29 amino acid residues in length
  MISC_FEATURE (2)..(2): Xaa denotes a basic amino acid residue
  MISC_FEATURE (9)..(9): Xaa denotes methionine or a structural derivative thereof
  MISC_FEATURE (11)..(12): Xaa denotes glycine, D-proline or L-proline
  MISC_FEATURE (15)..(15): Xaa denotes a peptide of 0 to 5 amino acid residues in length
XXGAIIGLXVXXVVX

SEQ ID NO: 66 - DNA, 20 bp, Artificial Sequence
  Synthetic oligonucleotide
gcaccttctttcccttcatc

SEQ ID NO: 67 - DNA, 20 bp, Artificial Sequence
  Synthetic oligonucleotide
tgctgatgtaccagttgggg

SEQ ID NO: 68 - DNA, 19 bp, Artificial Sequence
  Synthetic oligonucleotide
cagtccttgacctgcgacc

SEQ ID NO: 69 - DNA, 19 bp, Artificial Sequence
  Synthetic oligonucleotide
gcctcacatcacatccttg

(19) United States
(12) Patent Application Publication
(10) Pub. No.: US 2007/0213512 A1
(43) Pub. Date: Sep. 13, 2007

(54) Amyloid beta peptide analogs and assemblies thereof

(76) Inventors: Grant A. Krafft, Glenview, IL (US); William L. Klein, Winnetka, IL (US); Kirsten L. Viola, Chicago, IL (US); Mary P. Lambert, Glenview, IL (US); Todd R. Pray, San Mateo, CA (US); Ray Lowe, San Francisco, CA (US)

Correspondence Address: LICATA & TYRRELL P.C., 66 E. MAIN STREET, MARLTON, NJ, 08053, US

(21) Appl. No.: 11/686,570

(22) Filed: Mar. 15, 2007

Related U.S. Application Data
(63) Continuation-in-part of application No. 11/569,122.
(63) Continuation-in-part of application No. 11/142,869, filed on Jun. 1, 2005.
Application No. 11/142,869 is a continuation of application No. 10/924,372, filed on Aug. 23, 2004, now abandoned.
Application No. 10/924,372 is a continuation of application No. 10/676,871, filed on Oct. 1, 2003, now abandoned.
(60) Provisional application No. 60/829,863, filed on Oct. 17, 2006.
(60) Provisional application No. 60/782,742, filed on Mar. 15, 2006.
(60) Provisional application No. 60/636,466, filed on Dec. 15, 2004.
(60) Provisional application No. 60/571,267, filed on May 14, 2004.
(60) Provisional application No. 60/415,074, filed on Oct. 1, 2002.

(51) Int. Cl. C07K 14/00

(52) U.S. Cl. 530/395

(57) ABSTRACT

The present invention provides amyloid β peptide assemblies composed of at least three amyloid β peptide subunits, wherein at least one of the amyloid β peptide subunits is an amyloid β peptide analog. The invention further relates to metal complexes of amyloid β peptide assemblies and the use of amyloid β peptide assemblies as vaccines and in the identification of agents that modulate assembly of the amyloid β peptide subunits.

### INTRODUCTION

This application is a continuation-in-part application of U.S. Provisional Patent Application Ser. No. 60/829,863, filed Oct. 17, 2006; U.S. Provisional Patent Application Ser. No. 60/782,742, filed Mar. 15, 2006; U.S. patent application Ser. No. 11/569,122, filed Jul. 5, 2005, which claims the benefit of priority from PCT/US2005/017176, filed May 16, 2005, U.S. Provisional Patent Application Ser. No. 60/636,466, filed Dec. 15, 2004, and U.S. Provisional Patent Application Ser. No. 60/571,267, filed May 14, 2004; U.S. patent application Ser. No. 11/142,869, filed Jun. 1, 2005, which is a continuation of U.S. patent application Ser. No. 10/924,372, filed Aug. 23, 2004, which is a continuation of U.S. patent application Ser. No. 10/676,871, filed Oct. 1, 2003, which claims the benefit of priority from U.S. Provisional Patent Application Ser. No. 60/415,074, filed Oct. 1, 2002, the contents of which are incorporated herein by reference in their entireties.

This invention was made in the course of research sponsored by the National Institutes of Health. The U.S. government may have certain rights in this invention.

### BACKGROUND OF THE INVENTION

Alzheimer's disease (AD) is a progressive and degenerative dementia (Terry, et al. (1991) Ann. Neurol. 30(4):572-80; Coyle (1987) in Encyclopedia of Neuroscience, Ed. G. Adelman, pp. 29-31, Birkhäuser: Boston-Basel-Stuttgart), which in its early stages manifests primarily as a profound inability to form new memories (Selkoe (2002) Science 298(5594):789-91). AD was first described nearly a century ago by German psychiatrist Alois Alzheimer, who identified prominent neuritic plaques and neurofibrillary tangles as the major pathology in brain tissue samples taken at autopsy from a demented patient. Subsequently, the amyloid β (Aβ) peptide was discovered and shown to be the major protein constituent of amyloid plaques and cerebrovascular amyloid deposits. Additional research identified the amyloid precursor protein (APP) gene, the secretase enzymes that cleave amyloid β from APP, and demonstration that amyloid β could be directly toxic to cultured neurons.

The role of amyloid β in AD has been described as “a slowly evolving cascade in which excessive deposition of Aβ plays an early and critical role” (Selkoe (1991) Neuron 6(4):487-98), and this concept was formalized as the “amyloid cascade hypothesis” (Hardy & Higgins (1992) Science 256(5054):184-5). The seminal events leading to AD were amyloid β deposition and subsequent fibril-induced neuronal death. Key supporting evidence for this hypothesis emerged from studies of various presenilin and APP gene mutations linked to early-onset familial AD, all of which led to a single common biochemical consequence, elevated production of amyloid β 1-42.

Amyloid β 1-42 is quite hydrophobic and rapidly assembles into fibrils. Although it only represents 10-15% of the total amyloid β peptide production, it is the predominant peptide in plaques, accompanied by smaller quantities of amyloid β 1-43 and N-terminal truncated forms of amyloid β 1-42 and amyloid β 1-43. Relatively little amyloid β 1-40 deposits in plaques, due to its higher solubility, but it does assemble into fibrils at micromolar to millimolar concentrations in vitro. Fibrils prepared from synthetic amyloid β 1-40 or amyloid β 1-42 exhibit morphologies and Congo red birefringence similar to AD fibril deposits and both peptides can be toxic to neurons in culture. A number of studies have demonstrated that amyloid β neurotoxicity requires prior assembly into fibrils (Lorenzo, & Yankner (1994) Proc. Natl. Acad. Sci. USA 91(25):12243-7) and several reports have described trophic or cognition enhancing properties of the amyloid β 1-40 at nanomolar concentrations. The link between fibrils and in vitro neurotoxicity was sufficient to convince many AD researchers that amyloid plaques were the cause of AD.

Despite the supporting evidence and intuitive appeal of the amyloid cascade hypothesis, a number of clinical and pathology studies suggest that plaques and fibrils are not responsible for cognitive deficits in AD. For example, careful analysis of plaque number and location revealed little or no correlation with nerve cell loss and cognitive impairment (Terry, et al. (1991) Ann. Neurol. 30(4):572-80; Terry, et al. (1999) “Alzheimer Disease”, 2^{nd} Edition, Lippincott Williams & Wilkins: Philadelphia, Pa.; McLean, et al. (1999) Ann. Neurol. 46(6):860-6; Hibbard & McKeel, Jr. (1997) Anal. Quant. Cytol. Histol. 19(2):123-38; Sze, et al. (1997) J. Neuropathol. Exp. Neurol. 56(8):933-44). A possible explanation for such plaque-independent functional deficits has been suggested as slowly sedimenting amyloid β which enhances the neurotoxicity of amyloid β deposits in AD brains (Oda, et al. (1995) Exp Neurol. 136(1):22-31). However, the nature of the slowly sedimenting amyloid β was not described.

### SUMMARY OF THE INVENTION

The present invention is an isolated soluble amyloid β peptide assembly composed of at least three amyloid β peptide subunits, wherein at least one subunit is an amyloid β X-Y peptide analog, wherein X is an integer between 1 and 28, and Y is 40, 42 or 43; and wherein when X is 1, the amino acid residue at position 1 is an aspartic acid or a modified amino acid residue. In one embodiment, the analog further includes a label. In another embodiment, the analog further includes one or two additional carboxyl-terminal amino acid residues. In a further embodiment, the analog has the amino acid sequence R₂-Xaa₁-Gly-Ala-Ile-Ile-Gly-Leu-Xaa₂-Val-Xaa₃-Xaa₄-Val-Val-R₃ (SEQ ID NO:65), wherein R₂ denotes a peptide of 0 to 29 amino acid residues in length; Xaa₁ is a basic amino acid residue, Xaa₂ is methionine or a structural derivative thereof; Xaa₃ and Xaa₄ are independently glycine, D-proline or L-proline, with the proviso that both Xaa₃ and Xaa₄ are not simultaneously a proline; and R₃ is a peptide of 0 to 5 amino acid residues in length. Still other embodiments embrace assemblies with one or more metal atoms and assemblies produced from a homogenous or heterogenous population of amyloid β peptide subunits.

### BRIEF DESCRIPTION OF THE DRAWINGS

FIG. 1 shows the characterization of biotin labeled amyloid β peptide assemblies. Biotinylated amyloid β peptide assemblies (b-ADDLs) appear in low molecular weight (LMW) peak.

FIG. 2 is a graph of amyloid β peptide assembly concentration measured as amyloid β 1-42 concentration (nM) vs. % dead cells for brain slices from mice treated with the amyloid β peptide assembly preparations.

FIG. 3 is a graph of amyloid β concentration (μM) versus activated glia (number) obtained upon incubation of astrocytes with amyloid β peptide assemblies or amyloid β 17-42.

FIG. 4 is a graph showing that Cu²⁺ can induce rapid kinetic assembly of amyloid β peptides in a homogenous fluorescence quenching assay.

FIG. 5 is a graph of a SUPERDEX-75 elution profile showing intermediate molecular weight (IMW) amyloid β peptide 1-42 oligomers formed in the presence of CuCl₂ and SDS.

FIG. 6 is a graph comparing amyloid β 1-42 peptide, amyloid β 1-43 peptide and amyloid β 1-40 peptide oligomerization at higher concentration, lower temperature and shorter time.

FIG. 7 is a graph of a SUPERDEX-75 elution profile showing that the amyloid β 1-42 [Nle35-DPro37] peptide assembled in CuCl₂ remains in the LMW peak.

FIG. 8 shows the quantification of binding of metal complexes of amyloid β 1-42 and amyloid β 1-43 peptide assemblies to primary hippocampal neurons (20C2 detection).

### DETAILED DESCRIPTION OF THE INVENTION

The present invention relates to soluble amyloid β peptide structures that are neurotoxic. Based upon several unique structural features of the amyloid β peptide, namely a beta turn flanked by hydrophobic amino acid residues, amyloid β peptide subunits readily assemble into these soluble oligomeric structures. Using novel methods, soluble amyloid β peptide assemblies have been generated in vitro which lack fibrillar structures. In heterogeneous samples, the removal of the larger, fibrillar forms of amyloid by centrifugation does not remove these soluble, neurotoxic, amyloid β peptide assemblies in the supernatant fractions. These novel neurotoxic, soluble, oligomeric forms are referred to herein as soluble amyloid β peptide assemblies, amyloid-derived dementing ligands, or amyloid β-derived diffusible ligands (ADDLs). The finding that soluble amyloid β peptide assemblies are neurotoxic is particularly unexpected since conventional thinking suggests that it is fibril structures that constitute the toxic form of amyloid β (Lorenzo, et al. (1994) Proc. Natl. Acad. Sci. USA 91:12243-12247; Howlett, et al. (1995) Neurodegen. 4:23-32).

Thus, the present invention provides an isolated soluble non-fibrillar amyloid β peptide assembly (i.e., ADDL) composed of at least three amyloid β peptide subunits. In contrast to monomeric and dimeric amyloid β, the soluble amyloid β peptide assemblies of the invention are neurotoxic, e.g., as determined in neuronal cell cultures or in the organotypic brain slice cultures. For the purposes of the present invention, an amyloid β peptide assembly is said to be soluble in that it remains in solution and is not removed from the solution by methods such as centrifugation. In other words, an amyloid β peptide assembly is not an aggregate or fibril which can be sedimented by centrifugation.

An assembly of the present invention is isolated in the sense that it is substantially purified or essentially free from components that normally accompany or interact with the assembly. Thus, an isolated assembly is substantially free of other cellular material or substantially free of precursors, fibrillar forms of amyloid β, as well as aggregates of amyloid β.

An amyloid β peptide assembly is defined as a multisubunit structure or complex formed by the association of at least three subunits of an amyloid β peptide. In certain embodiments, an amyloid β peptide assembly is composed of more than three amyloid β peptide subunits. In this regard, the present invention embraces an amyloid β peptide assembly composed of three to about six amyloid β peptide subunits, three to about 12 amyloid β peptide subunits, three to about 24 amyloid β peptide subunits, or three to about 48 amyloid β peptide subunits. In other embodiments, the amyloid β peptide assembly of the present invention is composed of three to about 12 amyloid β peptide subunits, 13 to about 24 amyloid β peptide subunits, or 25 to about 48 amyloid β peptide subunits. In further embodiments, the amyloid β peptide assembly of the present invention is composed of at least three, at least 13, at least 25, or at least 48 amyloid β peptide subunits. In particular, the invention provides an isolated amyloid β peptide assembly, wherein the assembly is desirably a trimer, tetramer, pentamer, hexamer, heptamer, octomer, nonamer, decamer, etc. It is further contemplated that depending upon the method employed for detecting amyloid β peptide assemblies, assemblies of more than 48 amyloid β peptide subunits are possible and are therefore embraced by the present invention.

Desirably, the isolated amyloid β peptide assembly according to the invention is composed of globules ranging in size from about 4.7 nm to about 6.2 nm as measured by atomic force microscopy. In certain embodiments, the isolated amyloid β peptide assembly is composed of globules ranging in size from about 4.9 nm to about 5.4 nm and globules ranging in size from about 5.7 nm to about 6.2 nm, as measured by atomic force microscopy. In particular embodiments, the isolated amyloid β peptide assembly according to the invention is composed of from about 30% to about 85%, or more desirably from about 40% to about 75%, of two predominant forms of globules, namely, globules ranging in size from about 4.9 nm to about 5.4 nm, and globules ranging in size from about 5.7 nm to about 6.2 nm, as measured by atomic force microscopy. However, it is also desirable that the peptide assembly is composed of globules ranging in size from about 5.3 to about 5.7 nm.

By non-denaturing gel electrophoresis, bands corresponding to amyloid β peptide assemblies run at about from 26 kD to about 28 kD, with a separate broad band representing sizes of from about 36 kD to about 108 kD. Under denaturing conditions (e.g., on a 15% SDS-polyacrylamide gel), the amyloid β peptide assemblies are observed as a band that runs at from about 22 kD to about 24 kD, which can further include a band that runs at about 18 to about 19 kD.

Accordingly, the present invention provides an isolated soluble non-fibrillar amyloid β peptide assembly (i.e., ADDL) that has a molecular weight of from about 26 kD to about 28 kD as determined by non-denaturing gel electrophoresis. The invention also provides an isolated soluble non-fibrillar amyloid β peptide assembly that runs as a band corresponding to a molecular weight of from about 22 kD to about 24 kD as determined by electrophoresis on a 15% SDS-polyacrylamide gel. The invention further provides an isolated soluble non-fibrillar amyloid β peptide assembly that runs as a band corresponding to a molecular weight of from about 18 kD to about 19 kD as determined by electrophoresis on a 15% SDS-polyacrylamide gel.

Furthermore, using a 16.5% Tris-tricine SDS-polyacrylamide gel system, additional amyloid β peptide assembly bands can be visualized. The increased resolution provided by this gel system confirms the ability to obtain an isolated oligomeric structure having a molecular weight ranging from about 13 kD to about 116 kD, as determined by electrophoresis on a 16.5% Tris-tricine SDS-polyacrylamide gel. The bands appear to correspond to distinct oligomeric species of amyloid β peptide assemblies. In particular, use of this gel system allows visualization of bands corresponding to trimer with a size of about 13 to about 14 kD, tetramer with a size of about 17 to about 19 kD, pentamer with a size of about 22 kD to about 23 kD, hexamer with a size of about 26 to about 28 kD, heptamer with a size from about 32 kD to 33 kD, and octamer with a size from about 36 kD to about 38 kD, as well as larger soluble oligomers ranging in size from about 12 monomers to about 24 monomers. Thus, the present invention further provides an isolated amyloid β peptide assembly, wherein the assembly has, as determined by electrophoresis on a 16.5% Tris-tricine SDS-polyacrylamide gel, a molecular weight of from about 13 kD to about 14 kD, from about 17 kD to about 19 kD, from about 22 kD to about 23 kD, from about 26 kD to about 28 kD, from about 32 kD to about 33 kD, or from about 36 kD to about 38 kD.

Assemblies of the present invention can be produced by any suitable method. For example, is has now been found that when monomeric amyloid β 1-42 peptide solutions are maintained at low temperature in an appropriate media, formation of sedimentable amyloid β fibrils is almost completely blocked. Amyloid β peptide, however, does self-associate in these low-temperature solutions, forming assemblies essentially indistinguishable from those chaperoned by clusterin. Furthermore, amyloid β peptide assemblies also form when monomeric amyloid β peptide solutions are incubated at 37° C. in brain slice culture medium but at very low concentration (e.g., ≦50 nM), indicating a potential to form under physiological conditions. In addition, the assembly can be accelerated by the presence of metal ions. Independent of the method employed, amyloid β peptide assemblies are relatively stable and show no conversion to fibrils during a 24-hour tissue culture experiment.

Accordingly, the assemblies of the present invention can be formed in vitro with or, in particular embodiments, without exogenous cross-linking agents. When a solution (e.g., a DMSO solution) containing monomeric amyloid β peptide is diluted into cold tissue culture media (e.g., F12 cell culture media) to a final concentration ranging from about 5 nM to 500 μM, then allowed to incubate at about 4° C. for about 2 to about 48 hours and centrifuged for about 5 minutes to one hour at about 14,000×g at a temperature of 4° C., the supernatant fraction contains small, soluble oligomeric assemblies that are highly neurotoxic, e.g., in neuronal cell and brain slice cultures. The amyloid β peptide assemblies also can be formed by coincubation of amyloid β peptide with certain appropriate agents, e.g., clusterin (a senile plaque protein that also is known as ApoJ).

In addition to F12 cell culture media, other suitable tissue culture media can be used to support or facilitate the assembly of the amyloid β peptide subunits. For example a substitute F12 medium can be employed which contains the following components: N,N-dimethylglycine, D-glucose, calcium chloride, copper sulfate pentahydrate, iron(II) sulfate heptahydrate, potassium chloride, magnesium chloride, sodium chloride, sodium bicarbonate, disodium hydrogen phosphate, and zinc sulfate heptahydrate.

In particular, synthetic F12 media can be employed. Such media is composed of: N,N-dimethylglycine (from about 600 to about 850 mg/L), D-glucose (from about 1.0 to about 3.0 g/L), calcium chloride (from about 20 to about 40 mg/L), copper sulfate pentahydrate (from about 15 to about 40 mg/L), iron(II) sulfate heptahydrate (from about 0.4 to about 1.2 mg/L), potassium chloride (from about 160 to about 280 mg/L), magnesium chloride (from about 40 to about 75 mg/L), sodium chloride (from about 6.0 to about 9.0 g/L), sodium bicarbonate (from about 0.75 to about 1.4 g/L), disodium hydrogen phosphate (from about 120 to about 160 mg/L), and zinc sulfate heptahydrate (from about 0.7 to about 1.1 mg/L). Optimally, the synthetic F12 media exemplified herein is employed. Further, the pH of the F12 media is desirably adjusted, for instance using 0.1 M sodium hydroxide, to a pH of from about 7.0 to about 8.5, and most desirably a pH of about 8.0.

Amyloid β peptide subunits of use in accordance with the present invention include wild-type human amyloid β peptides (i.e., amyloid β 1-43, amyloid β 1-42, and amyloid β 1-40), amyloid β peptides with truncated amino-termini (i.e., amyloid β X-43, amyloid β X-42, and amyloid β X-40), and in particular, analogs of wild-type and/or truncated amyloid β peptides. Such amyloid β peptide subunits can be chemically synthesized or recombinantly produced using conventional technologies well-known to those skilled in the art. As used herein, a peptide is an organic compound composed of amino acids linked together chemically by peptide bonds. Peptides of the present invention generally range in size from about 13 amino acid residues to about 50 amino acid residues or more particularly 13 to 47 amino acid residues in length. In this regard, particular embodiments embrace an amyloid β peptide of X-Y length, wherein “X” is an integer between 1 and 28 and “Y” is 40, 42 or 43. As such, the present invention includes an amyloid β X-43 peptide, an amyloid β X-42 peptide, and an amyloid β X-40 peptide. Alternatively stated, an amyloid β peptide, such as amyloid β 1-42, amyloid β 1-43, or amyloid β 1-40, can be missing one, two, three, four, five, six, seven, eight, nine, ten, 11, 12, 13, 14, 15, 16, 17, 18, 19, 20, 21, 22, 23, 24, 25, 26, or 27 amino-terminal amino acid residues.

As the skilled artisan can appreciate, the location of amino acid residues referred to herein is with reference to the wild-type amyloid β peptide sequence. By way of illustration, the amino acid sequence of wild-type amyloid β 1-43 peptide is:

(SEQ ID NO:1)

```
Asp-Ala-Glu-Phe-Arg-His-Asp-Ser-Gly-Tyr-Glu-Val-
  1 2 3 4 5 6 7 8 9 10 11 12
His-His-Gln-Lys-Leu-Val-Phe-Phe-Ala-Glu-Asp-Val-
  13 14 15 16 17 18 19 20 21 22 23 24
Gly-Ser-Asn-Lys-Gly-Ala-Ile-Ile-Gly-Leu-Met-Val-
  25 26 27 28 29 30 31 32 33 34 35 36
Gly-Gly-Val-Val-Ile-Ala-Thr
  37 38 39 40 41 42 43
```

Within this context, a truncated wild-type amyloid β X-43 peptide, wherein X is 4, would lack amino acid residues 1 through 3, i.e., Asp-Ala-Glu. Similarly, the lysine residue at position 16 will be referred to as position 16 whether, e.g., an amyloid β 1-43 peptide or an amyloid β 7-43 peptide is being discussed.

Having identified the structural features required for assembly of amyloid β peptide subunits into oligomers, the present invention specifically provides analogs of amyloid β peptide. For the purposes of the present invention, an amyloid β peptide analog is defined as a structural derivative of a parent amyloid β peptide, wherein the structural derivative retains the ability to associate with other amyloid β peptides and form a soluble amyloid β peptide assembly. In particular embodiments, the analog is also biologically active in that it continues to possess the neurotoxic activity of the parent peptide when in a soluble amyloid β peptide assembly.

Amyloid β peptide analogs of the present invention generally contain one or more amino acid substitutions. An amino acid substitution refers to the replacement of at least one existing amino acid residue in a predetermined sequence with another, different “replacement” amino acid residue. An amyloid β peptide analog of the present invention can differ from the parent or wild-type amyloid β peptide by as few as 1, 2, 3, 4, 5, or 6 amino acid residue substitutions or may have as many as 32 amino acid substitutions (e.g., substitutions at amino acid residues 1-27, 35, 37 or 38, and 41-43). In particular embodiments, an analog of the present invention has one or more amino acid substitutions located at residues 1 to 22, 35, 37, 38, or 41 to 43. In one embodiment, the replacement amino acid residue is a standard, levorotatory (L-) amino acid residue (see Table 1). In another embodiment, the replacement amino acid residue is a modified amino acid residue as in Table 2 or modified by post-translation modification. In particular embodiments, when X of an amyloid β X-Y peptide is 1, the amino acid residue at position 1 is an aspartic acid or a modified amino acid residue.

TABLE 1

Abbreviation

| | Three-Letter | One-Letter |
|---|---|---|
| Amino Acid Residue | Code | Code |
| Alanine | Ala | A |
| Arginine | Arg | R |
| Asparagine | Asn | N |
| Aspartic acid (Aspartate) | Asp | D |
| Cysteine | Cys | C |
| Glutamine | Gln | Q |
| Glutamic acid (Glutamate) | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The term “modified amino acid residue” is used herein to denote an amino acid residue which is not naturally incorporated into a polypeptide chain during protein biosynthesis, i.e., during translation. In this regard, a modified amino acid residue is not proteinogenic or a standard amino acid. Modified amino acid residues include those set forth in Table 2 as well as any other non-protein amino acids known in the art, including amino acid residues modified by post-translation modification (e.g., acetylation, amidation, formylation, hydroxylation, methylation, phosphorylation, or sulfatation).

TABLE 2

| | |
|---|---|
| Modified Amino Acid Residue | Abbreviation |

Amino Acid Residue Derivatives

| | |
|---|---|
| 2-Aminoadipic acid | Aad |
| 3-Aminoadipic acid | bAad |
| beta-Alanine, beta-Aminoproprionic acid | bAla |
| 2-Aminobutyric acid | Abu |
| 4-Aminobutyric acid, Piperidinic acid | 4Abu |
| 6-Aminocaproic acid | Acp |
| 2-Aminoheptanoic acid | Ahe |
| 2-Aminoisobutyric acid | Aib |
| 3-Aminoisobutyric acid | bAib |
| 2-Aminopimelic acid | Apm |
| t-butylalanine | t-BuA |
| Citrulline | Cit |
| Cyclohexylalanine | Cha |
| 2,4-Diaminobutyric acid | Dbu |
| Desmosine | Des |
| 2,2′-Diaminopimelic acid | Dpm |
| 2,3-Diaminoproprionic acid | Dpr |
| N-Ethylglycine | EtGly |
| N-Ethylasparagine | EtAsn |
| Homoarginine | hArg |
| Homocysteine | hCys |
| Homoserine | hSer |
| Hydroxylysine | Hyl |
| Allo-Hydroxylysine | aHyl |
| 3-Hydroxyproline | 3 Hyp |
| 4-Hydroxyproline | 4 Hyp |
| Isodesmosine | Ide |
| allo-Isoleucine | aIle |
| Methionine sulfoxide | MSO |
| N-Methylglycine, sarcosine | MeGly |
| N-Methylisoleucine | MeIle |
| 6-N-Methyllysine | MeLys |
| N-Methylvaline | MeVal |
| 2-Naphthylalanine | 2-Nal |
| Norvaline | Nva |
| Norleucine | Nle |
| Ornithine | Orn |
| 4-Chlorophenylalanine | Phe (4-Cl) |
| 2-Fluorophenylalanine | Phe (2-F) |
| 3-Fluorophenylalanine | Phe (3-F) |
| 4-Fluorophenylalanine | Phe (4-F) |
| Phenylglycine | Phg |
| Beta-2-thienylalanine | Thi |

Dextrorotary (D-) Amino Acid Residues

| | |
|---|---|
| D-Alanine | D-ala |
| D-Arginine | D-arg |
| D-Asparagine | D-Asn |
| D-Aspartic acid (D-Aspartate) | D-Asp |
| D-Cysteine | D-Cys |
| D-Glutamine | D-Gln |
| D-Glutamic acid (D-Glutamate) | D-Glu |
| D-Glycine | D-Gly |
| D-Histidine | D-His |
| D-Isoleucine | D-Ile |
| D-Leucine | D-Leu |
| D-Lysine | D-Lys |
| D-Methionine | D-Met |
| D-Phenylalanine | D-Phe |
| D-Proline | D-Pro |
| D-Serine | D-Ser |
| D-Threonine | D-Thr |
| D-Tryptophan | D-Trp |
| D-Tyrosine | D-Tyr |
| D-Valine | D-Val |

In certain embodiments, the modified amino acid residue is reactive and/or photoreactive. For example, photoreactive p-benzoyl-L-phenylalanine (Bpa) can be incorporated into amyloid β peptide subunits using solid-phase synthesis methods. See, e.g., Kauer, et al. (1986) J. Biol. Chem. 261:10695-10700. Desirably, reactive or photoreactive groups are incorporated at the N-terminus of the amyloid β peptide, incorporated as a side-chain of glycine placed within the amyloid β peptide sequence, incorporated on the ε-amino group of a lysine within the peptide sequence (e.g., lysine 16 or lysine 28), or incorporated at a lysine substitution located at the N-terminus of the amyloid β peptide. Photoreactive amino acid derivatives thus incorporated into the amyloid β peptide subunit can function to cross-link the individual subunits to one another or cross-link the instant assemblies to one or more receptors.

In particular embodiments, an amyloid β peptide analog of the invention has one to ten, one to five, one to four, or desirably one to three modified amino acid residues incorporated into the peptide sequence. By way of illustration, the amyloid β peptide analog can contain any combination of an ornithine at position 1, 3, 7, and/or position 11; Bpa at position 10; norleucine at position 35; and/or D-proline at position 37.

In further embodiments, an amyloid β peptide or amyloid β peptide analog of the invention is labeled. A labeled amyloid β peptide or analog is an amyloid β peptide which contains at least one detectable moiety, e.g., detectable by fluorescence, luminescence, spectrometry, and the like. For example, a labeled amyloid β peptide can have a fluorophore e.g., fluorescein) attached by conventional methods at the omega amino moiety of a lysine side chain (e.g., the lysine located at amino acid position 16 or 28; or alternatively a lysine introduced by amino acid substitution). Other suitable labels include, but are not limited to, biotin/avidin/streptavidin, radioisotopes, enzyme-substrates, and the like. Labeled amyloid proteins and peptides are described, e.g., in U.S. Pat. Nos. 5,200,339; 5,434,050; 5,721,106; and 5,837,473. Labels can be incorporated at the N-terminus of the amyloid β peptide (e.g., biotin can be attached to the N-terminus using an activated ester of biotin, biotin-ONp), incorporated as a side-chain of glycine placed within the amyloid β peptide sequence, incorporated on the omega amino group of a lysine within the peptide sequence (e.g., lysine 16 or lysine 28), incorporated at a lysine substitution located at the N-terminus of the amyloid β peptide, or incorporated at any other suitable amino acid residue as disclosed herein or routinely modified in the art.

The addition of one or more additional amino acid residues to the amino-terminus (N-terminus) and/or carboxyl-terminus (C-terminus) of the amyloid β peptide is also embraced by the present invention. For example, a cysteine residue can be added to the N-terminus to provide a free thiol group for conjugation of a label or other moiety, e.g., maleimide PEG. Similarly, the amyloid β peptide can be modified to have one or two extra amino acid residues at the C-terminus. Amino acid residues added to the N-terminus and/or C-terminus of the amyloid β peptide can be wild-type levorotatory (L-) amino acid residues (see Table 1), or modified amino acid residues as disclosed herein.

Wild-type human amyloid β 1-43 peptide is set forth herein as SEQ ID NO:1. By way of illustration, Table 3 provides exemplary analogs of human amyloid β 1-43 peptide.

TABLE 3

| | |
|---|---|
| | SEQ |
| | ID |
| Peptide sequence | NO: |

| | |
|---|---|
| Asp-Ala-Glu-Phe-Asp-His-Arg-Ser-Gly-Tyr-Glu- | 2 |
| Val-His-Ser-Gln-Lys-Leu-Val-Phe-Phe-Ala-Glu- | |
| Asp-Val-Gly-Ser-Asn-Lys-Gly-Ala-Ile-Ile-Gly- | |
| Leu-Met-Val-Gly-Gly-Val-Val-Ile-Ala-Thr | |
| Asp-Ala-Glu-Phe-Arg-Asp-His-Ser-Gly-Tyr-His- | 3 |
| Val-Glu-His-Gln-Lys-Leu-Val-Phe-Phe-Ala-Glu- | |
| Asp-Val-Gly-Ser-Asn-Lys-Gly-Ala-Ile-Ile-Gly- | |
| Leu-Met-Val-Gly-Gly-Val-Val-Ile-Ala-Thr | |
| Asp-Ala-Glu-Phe-Arg-His-Asp-Ser-Gly-Tyr-Glu- | 4 |
| Val-His-His-Gln-Lys-Leu-Val-Phe-Phe-Ala-Glu- | |
| Asp-Val-Gly-Ser-Asn-Lys-Gly-Ala-Ile-Ile-Gly- | |
| Leu-Met-Val-Gly-Gly-Val-Val-Ile-Ala-Ala | |
| Asp-Ala-Glu-Phe-Arg-His-Asp-Ser-Gly-Tyr-Glu- | 5 |
| Val-His-His-Gln-Lys-Leu-Val-Phe-Phe-Ala-Glu- | |
| Asp-Val-Gly-Ser-Asn-Lys-Gly-Ala-Ile-Ile-Gly- | |
| Leu-Met-Val-Gly-Gly-Val-Val-Ile-Ala-Val | |
| Asp-Ala-Glu-Phe-Arg-His-Asp-Ser-Gly-Tyr-Glu- | 6 |
| Val-His-His-Gln-Lys-Leu-Val-Phe-Phe-Ala-Glu- | |
| Asp-Val-Gly-Ser-Asn-Lys-Gly-Ala-Ile-Ile-Gly- | |
| Leu-Met-Val-Gly-Gly-Val-Val-Ile-Ala-Leu | |
| Asp-Ala-Glu-Phe-Arg-His-Asp-Ser-Gly-Tyr-Glu- | 7 |
| Val-His-His-Gln-Lys-Leu-Val-Phe-Phe-Ala-Glu- | |
| Asp-Val-Gly-Ser-Asn-Lys-Gly-Ala-Ile-Ile-Gly- | |
| Leu-Met-Val-Gly-Gly-Val-Val-Ile-Ala-Ile | |
| Asp-Ala-Glu-Phe-Arg-His-Asp-Ser-Gly-Tyr-Glu- | 8 |
| Val-His-His-Gln-Lys-Leu-Val-Phe-Phe-Ala-Glu- | |
| Asp-Val-Gly-Ser-Asn-Lys-Gly-Ala-Ile-Ile-Gly- | |
| Leu-Nle-Val-Pro*-Gly-Val-Val-Ile-Ala-Thr | |
| Lys#-Ala-Glu-Phe-Arg-His-Asp-Ser-Gly-Tyr-Glu- | 9 |
| Val-His-His-Gln-Lys-Leu-Val-Phe-Phe-Ala-Glu- | |
| Asp-Val-Gly-Ser-Asn-Lys-Gly-Ala-Ile-Ile-Gly- | |
| Leu-Met-Val-Gly-Gly-Val-Val-Ile-Ala-Thr | |
| Asp-Ala-Glu-Phe-Arg-His-Asp-Ser-Gly-Tyr-Glu- | 10 |
| Val-Asp-Glu-Gln-Lys-Leu-Val-Phe-Phe-Ala-Glu- | |
| Asp-Val-Gly-Ser-Asn-Lys-Gly-Ala-Ile-Ile-Gly- | |
| Leu-Met-Val-Pro*-Gly-Val-Val-Ile-Ser-Thr | |
| Asp-Ala-Glu-Phe-Arg-His-Asp-Ser-Gly-Tyr-Glu- | 11 |
| Val-Asp-Glu-Gln-Lys-Leu-Val-Phe-Phe-Ala-Glu- | |
| Asp-Val-Gly-Ser-Asn-Lys-Gly-Ala-Ile-Ile-Gly- | |
| Leu-Val-Val-Pro*-Gly Val-Val-Ile-Ser-Thr | |
| Glu-Val-Asp- | 12 |
| Glu-Gln-Lys-Leu-Val-Phe-Phe-Ala-Glu-Asp-Val- | |
| Gly-Ser-Asn-Lys-Gly-Ala-Ile-Ile-Gly-Leu-Met- | |
| Val-Pro*-Gly-Val-Val-Ile-Ser-Thr | |
| Glu-Val-Asp- | 13 |
| Glu-Gln-Lys-Leu-Val-Phe-Phe-Ala-Glu-Asp-Val- | |
| Gly-Ser-Asn-Lys-Gly-Ala-Ile-Ile-Gly-Leu-Val- | |
| Val-Pro*-Gly-Val-Val-Ile-Ser-Thr | |
| Asp-Ser-Gly-Tyr-Glu-Val-Gln- | 14 |
| Gln-Gln-Lys-Leu-Val-Phe-Phe-Ala-Glu-Asp-Val- | |
| Gly-Ser-Asn-Lys-Gly-Ala-Ile-Ile-Gly-Leu-Met- | |
| Val-Gly-Gly-Val-Val-Ile-Ala-Val | |
| Asp-Ser-Gly-Tyr-Glu-Val-Gln- | 15 |
| Gln-Gln-Gln-Leu-Val-Phe-Phe-Ala-Glu-Asp-Val- | |
| Gly-Ser-Asn-Lys-Gly-Ala-Ile-Ile-Gly-Leu-Met- | |
| Val-Gly-Gly-Val-Val-Ile-Ala-Val | |
| Asp-Ser-Gly-Phe-Glu-Val-Gln- | 16 |
| Gln-Gln-Lys-Leu-Val-Phe-Phe-Ala-Glu-Asp-Val- | |
| Gly-Ser-Asn-Lys-Gly-Ala-Ile-Ile-Gly-Leu-Met- | |
| Val-Pro*-Gly-Val-Val-Ile-Ala-Val | |
| Orn-Ser-Gly-Tyr-Orn-Val-Asp- | 17 |
| Asp-Gln-Glu-Leu-Val-Phe-Phe-Ala-Glu-Asp-Val- | |
| Gly-Ser-Asn-Lys-Gly-Ala-Ile-Ile-Gly-Leu-Nle- | |
| Val-Gly-Gly-Val-Val-Ile-Ala-Thr | |
| Leu-Val-Phe-Phe-Ala-Glu-Asp-Val-Gly-Ser- | 18 |
| Asn-Lys-Gly-Ala-Ile-Ile-Gly-Leu-Met-Val-Pro*- | |
| Gly-Val-Val-Ile-Ser-Thr | |
| Leu-Val-Phe-Phe-Ala-Glu-Asp-Val-Gly-Ser- | 19 |
| Asn-Lys-Gly-Ala-Ile-Ile-Gly-Leu-Val-Val-Pro*- | |
| Gly-Val-Val-Ile-Ser-Thr | |
| ‡Asp-Val-Gly-Ser- | 20 |
| Asn-Lys-Gly-Ala-Ile-Ile-Gly-Leu-Met-Val-Pro*- | |
| Gly-Val-Val-Ile-Ser-Thr | |
| ‡Asp-Val-Gly-Ser- | 21 |
| Asn-Lys-Gly-Ala-Ile-Ile-Gly-Leu-Val-Val-Pro*- | |
| Gly-Val-Val-Ile-Ser-Thr | |
| Lys-Gly-Ala-Ile-Ile-Gly-Leu-Met-Val-Pro*-Gly- | 22 |
| Val-Val-Ile-Ser-Thr | |
| Lys-Gly-Ala-Ile-Ile-Gly-Leu-Val-Val-Pro*-Gly- | 23 |
| Val-Val-Ile-Ser-Thr | |

Modified amino acid residues are underlined.
*Designates a D-amino acid residue.
#Designates biotinyl-L-lysine.
‡Designated acetylation.

Wild-type human amyloid β 1-42 peptide is set forth herein as SEQ ID NO:24. By way of illustration, Table 4 provides exemplary analogs of human amyloid β 1-42 peptide.

TABLE 4

| | SEQ |
|---|---|
| | ID |
| Peptide sequence | NO: |
| †Asp-Ala-Glu-Phe-Arg-His-Asp-Ser-Gly-Tyr-Glu- | 25 |
| Val-His-His-Gln-Lys-Leu-Val-Phe-Phe-Ala-Glu- | |
| Asp-Val-Gly-Ser-Asn-Lys-Gly-Ala-Ile-Ile-Gly- | |
| Leu-Met-Val-Gly-Gly-Val-Val-Ile-Ala | |
| Asp-Ala-Glu-Phe-Arg-Gly-Asp-Ser-Gly-Tyr-Glu- | 26 |
| Val-His-His-Gln-Lys-Leu-Val-Phe-Phe-Ala-Glu- | |
| Asp-Val-Gly-Ser-Asn-Lys-Gly-Ala-Ile-Ile-Gly- | |
| Leu-Met-Val-Gly-Gly-Val-Val-Ile-Ala | |
| \hat{ }Asp-Ala-Glu-Phe-Arg-His-Asp-Ser-Gly-Bpa-Glu- | 27 |
| Val-His-His-Gln-Lys-Leu-Val-Phe-Phe-Ala-Glu- | |
| Asp-Val-Gly-Ser-Asn-Lys-Gly-Ala-Ile-Ile-Gly- | |
| Leu-Met-Val-Gly-Gly-Val-Val-Ile-Ala | |
| Asp-Ala-Glu-Phe-Arg-His-Asp-Ser-Gly-Tyr-Glu- | 28 |
| Val-His-His-Gln-Lys-Leu-Val-Phe-Phe-Ala-Gly- | |
| Asp-Val-Gly-Ser-Asn-Lys-Gly-Ala-Ile-Ile-Gly- | |
| Leu-Met-Val-Gly-Gly-Val-Val-Ile-Ala | |
| Asp-Ala-Glu-Phe-Arg-His-Asp-Ser-Gly-Tyr-Glu- | 29 |
| Val-His-His-Gln-Lys-Leu-Val-Phe-Phe-Ala-Glu- | |
| Asp-Val-Gly-Ala-Asn-Lys-Gly-Ala-Ile-Ile-Gly- | |
| Leu-Met-Val-Gly-Gly-Val-Val-Ile-Ala | |
| Asp-Ala-Glu-Phe-Arg-His-Asp-Ser-Gly-Tyr-Glu- | 30 |
| Val-His-His-Gln-Lys-Leu-Val-Phe-Phe-Ala-Glu- | |
| Asp-Val-Gly-Ser-Ala-Lys-Gly-Ala-Ile-Ile-Gly- | |
| Leu-Met-Val-Gly-Gly-Val-Val-Ile-Ala | |
| Asp-Ala-Glu-Phe-Asp-His-Arg-Ser-Gly-Tyr-Glu- | 31 |
| Val-His-Ser-Gln-Lys-Leu-Val-Phe-Phe-Ala-Glu- | |
| Asp-Val-Gly-Ser-Asn-Lys-Gly-Ala-Ile-Ile-Gly- | |
| Leu-Met-Val-Gly-Gly-Val-Val-Ile-Ala | |
| Asp-Ala-Glu-Phe-Asp-His-Arg-Ser-Gly-Tyr-Glu- | 32 |
| Val-His-Ser-Gln-Lys-Leu-Val-Phe-Phe-Ala-Gly- | |
| Asp-Val-Gly-Ser-Asn-Lys-Gly-Ala-Ile-Ile-Gly- | |
| Leu-Met-Val-Gly-Gly-Val-Val-Ile-Ala | |
| Asp-Ala-Glu-Phe-Asp-His-Arg-Ser-Gly-Phe-Glu- | 33 |
| Val-His-Ser-Gln-Lys-Leu-Val-Phe-Phe-Ala-Gly- | |
| Asp-Val-Gly-Ser-Asn-Lys-Gly-Ala-Ile-Ile-Gly- | |
| Leu-Met-Val-Gly-Gly-Val-Val-Ile-Ala | |
| \hat{ }Asp-Ala-Glu-Phe-Asp-His-Arg-Ser-Gly-Tyr-Glu- | 34 |
| Val-His-Ser-Gln-Lys-Leu-Val-Phe-Phe-Ala-Glu- | |
| Asp-Val-Gly-Ser-Asn-Lys-Gly-Ala-Ile-Ile-Gly- | |
| Leu-Nle-Val-Pro*-Gly-Val-Val-Ile-Ala | |
| Asp-Ala-Glu-Phe-Arg-Asp-His-Ser-Gly-Tyr-Glu- | 35 |
| Val-His-His-Gln-Lys-Leu-Val-Phe-Phe-Ala-Glu- | |
| Asp-Val-Gly-Ser-Asn-Lys-Gly-Ala-Ile-Ile-Gly- | |
| Leu-Met-Val-Gly-Gly-Val-Val-Ile-Ala | |
| Asp-Ala-Glu-Phe-Arg-Asp-His-Ser-Gly-Tyr-Glu- | 36 |
| Val-His-His-Gln-Lys-Leu-Val-Phe-Phe-Ala-Gly- | |
| Asp-Val-Gly-Ser-Asn-Lys-Gly-Ala-Ile-Ile-Gly- | |
| Leu-Met-Val-Gly-Gly-Val-Val-Ile-Ala | |
| Asp-Ala-Glu-Phe-Arg-Asp-His-Ser-Gly-Tyr-His- | 37 |
| Val-Glu-His-Gln-Lys-Leu-Val-Phe-Phe-Ala-Glu- | |
| Asp-Val-Gly-Ser-Asn-Lys-Gly-Ala-Ile-Ile-Gly- | |
| Leu-Met-Val-Gly-Gly-Val-Val-Ile-Ala | |
| Asp-Ala-Glu-Phe-Arg-Asp-His-Ser-Gly-Phe-His- | 38 |
| Val-Glu-His-Gln-Lys-Leu-Val-Phe-Phe-Ala-Glu- | |
| Asp-Val-Gly-Ser-Asn-Lys-Gly-Ala-Ile-Ile-Gly- | |
| Leu-Met-Val-Gly-Gly-Val-Val-Ile-Ala | |
| Asp-Ala-Glu-Phe-Arg-Asp-His-Ser-Gly-Tyr-His- | 39 |
| Val-Glu-His-Gln-Lys-Leu-Val-Phe-Phe-Ala-Gly- | |
| Asp-Val-Gly-Ser-Asn-Lys-Gly-Ala-Ile-Ile-Gly- | |
| Leu-Met-Val-Gly-Gly-Val-Val-Ile-Ala | |
| Asp-Ala-Glu-Phe-Arg-His-Asp-Ser-Gly-Tyr-Glu- | 40 |
| Val-His-His-Gln-Lys-Leu-Val-Phe-Phe-Ala-Glu- | |
| Asp-Val-Gly-Ser-Asn-Lys-Gly-Ala-Ile-Ile-Gly- | |
| Leu-Met-Val-Pro*-Gly-Val-Val-Ile-Ala | |
| Asp-Ala-Glu-Phe-Arg-His-Asp-Ser-Gly-Tyr-Glu- | 41 |
| Val-His-His-Gln-Lys-Leu-Val-Phe-Phe-Ala-Glu- | |
| Asp-Val-Gly-Ser-Asn-Lys-Gly-Ala-Ile-Ile-Gly- | |
| Leu-Nle-Val-Pro*-Gly-Val-Val-Ile-Ala | |
| Asp-Ala-Glu-Phe-Arg-Asp-His-Ser-Gly-Tyr-His- | 42 |
| Val-Glu-His-Gln-Lys-Leu-Val-Phe-Phe-Ala-Glu- | |
| Asp-Val-Gly-Ser-Asn-Lys-Gly-Ala-Ile-Ile-Gly- | |
| Leu-Nle-Val-Pro*-Gly-Val-Val-Ile-Ala | |
| \hat{ }Asp-Ala-Glu-Phe-Arg-His-Asp-Ser-Gly-Tyr-Glu- | 43 |
| Val-His-His-Gln-Lys-Leu-Val-Phe-Phe-Ala-Glu- | |
| Asp-Val-Gly-Ser-Asn-Lys-Gly-Ala-Ile-Ile-Gly- | |
| Leu-Nle-Val-Pro*-Gly-Val-Val-Ile-Ala | |
| \hat{ }Asp-Ala-Glu-Phe-Arg-His-Asp-Ser-Gly-Bpa-Glu- | 44 |
| Val-His-His-Gln-Lys-Leu-Val-Phe-Phe-Ala-Glu- | |
| Asp-Val-Gly-Ser-Asn-Lys-Gly-Ala-Ile-Ile-Gly- | |
| Leu-Met-Val-Pro*-Gly-Val-Val-Ile-Ala | |
| Asp-Cys-Glu-Phe-Arg-His-Asp-Ser-Gly-Lys-Glu- | 45 |
| Val-His-His-Gln-Lys-Leu-Val-Phe-Phe-Ala-Glu- | |
| Asp-Val-Gly-Ser-Asn-Lys-Gly-Ala-Ile-Ile-Gly- | |
| Leu-Nle-Val-Gly-Gly-Val-Val-Ile-Ala | |
| Lys#-Ala-Glu-Phe-Arg-His-Asp-Ser-Gly-Tyr-Glu- | 46 |
| Val-His-His-Gln-Lys-Leu-Val-Phe-Phe-Ala-Glu- | |
| Asp-Val-Gly-Ser-Asn-Lys-Gly-Ala-Ile-Ile-Gly- | |
| Leu-Met-Val-Gly-Gly-Val-Val-Ile-Ala | |
| Orn-Ala-Orn-Phe-Glu-Asp-Orn-Ser-Gly-Tyr-Orn- | 47 |
| Val-Asp-Asp-Gln-Glu-Leu-Val-Phe-Phe-Ala-Glu- | |
| Asp-Val-Gly-Ser-Asn-Lys-Gly-Ala-Ile-Ile-Gly- | |
| Leu-Nle-Val-Gly-Gly-Val-Val-Ile-Ala | |
| \hat{ }Orn-Ala-Orn-Phe-Glu-Asp-Orn-Ser-Gly-Tyr-Orn- | 48 |
| Val-Asp-Asp-Gln-Glu-Leu-Val-Phe-Phe-Ala-Glu- | |
| Asp-Val-Gly-Ser-Asn-Lys-Gly-Ala-Ile-Ile-Gly- | |
| Leu-Nle-Val-Gly-Gly-Val-Val-Ile-Ala | |
| Ala-Glu-Phe-Arg-His-Asp-Ser-Gly-Tyr-Glu-Val- | 49 |
| His-His-Gln-Lys-Leu-Val-Phe-Phe-Ala-Gly-Asp- | |
| Val-Gly-Ser-Asn-Lys-Gly-Ala-Ile-Ile-Gly-Leu- | |
| Met-Val-Pro*-Gly-Val-Val-Ile-Ala | |
| Asp-Ser-Gly-Tyr-Glu-Val-Gln- | 50 |
| Gln-Gln-Lys-Leu-Val-Phe-Phe-Ala-Glu-Asp-Val- | |
| Gly-Ser-Asn-Lys-Gly-Ala-Ile-Ile-Gly-Leu-Met- | |
| Val-Gly-Gly-Val-Val-Ile-Ala | |
| Orn-Ser-Gly-Tyr-Orn-Val-Asp- | 51 |
| Asp-Gln-Glu-Leu-Val-Phe-Phe-Ala-Glu-Asp-Val- | |
| Gly-Ser-Asn-Lys-Gly-Ala-Ile-Ile-Gly-Leu-Nle- | |
| Val-Gly-Gly-Val-Val-Ile-Ala | |
| Orn-Ser-Gly-Phe-Orn-Val-Asp- | 52 |
| Asp-Gln-Glu-Leu-Val-Phe-Phe-Ala-Glu-Asp-Val- | |
| Gly-Ser-Asn-Lys-Gly-Ala-Ile-Ile-Gly-Leu-Nle- | |
| Val-Gly-Gly-Val-Val-Ile-Ala | |
| Val-Asp- | 53 |
| Asp-Gln-Gly-Leu-Val-Phe-Phe-Ala-Glu-Asp-Val- | |
| Gly-Ser-Asn-Lys-Gly-Ala-Ile-Ile-Gly-Leu-Nle- | |
| Val-Gly-Gly-Val-Val-Ile-Ala | |
| \hat{ }Val-Asp- | 54 |
| Asp-Gln-Glu-Leu-Val-Phe-Phe-Ala-Glu-Asp-Val- | |
| Gly-Ser-Asn-Lys-Gly-Ala-Ile-Ile-Gly-Leu-Nle- | |
| Val-Gly-Gly-Val-Val-Ile-Ala | |
| \hat{ }Asp-Orn-Asp-Orn- | 55 |
| Gly-Lys-Gly-Ala-Ile-Ile-Gly-Leu-Met-Val-Gly- | |
| Gly-Val-Val-Ile-Ala-Lys-Lys | |
| Leu-Val-Phe-Phe-Ala-Glu-Asp-Val-Gly-Ser- | 56 |
| Asn-Lys-Gly-Ala-Ile-Ile-Gly-Leu-Met-Val-Gly- | |
| Gly-Val-Val-Ile-Ala | |
| Leu-Val-Phe-Phe-Ala-Glu-Asp-Val-Gly-Ser- | 57 |
| Asn-Lys-Gly-Ala-Ile-Ile-Gly-Leu-Nle-Val-Gly- | |
| Gly-Val-Val-Ile-Ala | |
| \hat{ }Lys-Lys-Lys-Lys- | 58 |
| Gly-Lys-Gly-Ala-Ile-Ile-Gly-Leu-Met-Val-Gly- | |
| Gly-Val-Val-Ile-Ala | |

Modified amino acid residues are underlined.
†Designates N-terminal fluoroscein
*Designates a D-amino acid residue.
#Designates biotinyl-L-lysine.
\hat{ }Designates N-terminal biotinylation.

Wild-type human amyloid β 1-40 peptide is set forth herein as SEQ ID NO:59. By way of illustration, Table 5 provides exemplary analogs of human amyloid β 1-40 peptide.

TABLE 5

| | SEQ |
|---|---|
| | ID |
| Peptide sequence | NO: |
| †Asp-Ala-Glu-Phe-Arg-His-Asp-Ser-Gly-Tyr-Glu- | 60 |
| Val-His-His-Gln-Lys-Leu-Val-Phe-Phe-Ala-Glu- | |
| Asp-Val-Gly-Ser-Asn-Lys-Gly-Ala-Ile-Ile-Gly- | |
| Leu-Met-Val-Gly-Gly-Val-Val | |
| \hat{ }Asp-Ala-Glu-Phe-Arg-His-Asp-Ser-Gly-Bpa-Glu- | 61 |
| Val-His-His-Gln-Lys-Leu-Val-Phe-Phe-Ala-Glu- | |
| Asp-Val-Gly-Ser-Asn-Lys-Gly-Ala-Ile-Ile-Gly- | |
| Leu-Met-Val-Gly-Gly-Val-Val | |
| Asp-Cys-Glu-Phe-Arg-His-Asp-Ser-Gly-Lys-Glu- | 62 |
| Val-His-His-Gln-Lys-Leu-Val-Phe-Phe-Ala-Glu- | |
| Asp-Val-Gly-Ser-Asn-Lys-Gly-Ala-Ile-Ile-Gly- | |
| Leu-Nle-Val-Gly-Gly-Val-Val | |
| \hat{ }Leu-Val-Phe-Phe-Ala-Glu-Asp-Val-Gly-Ser- | 63 |
| Asn-Lys-Gly-Ala-Ile-Ile-Gly-Leu-Met-Val-Gly- | |
| Gly-Val-Val | |

Modified amino acid residues are underlined.
†Designates N-terminal fluoroscein
\hat{ }Designates N-terminal biotinylation.

As indicated, the assembly of the amyloid β peptide subunits into oligomers is facilitated by the presence of a beta turn flanked by hydrophobic amino acid residues. A peptide with these structural elements advantageously forms an internal C-terminal beta sheet, which enables the peptide to interact with other amyloid β peptide subunits and form oligomeric assemblies. Not wishing to be bound by theory, it is contemplated that the internal beta sheet lies in, e.g., a horizontal plane with the side chains of the hydrophobic amino acid residues alternating above and below the plane. In this regard, the side chains of multiple amyloid β peptide subunits can interdigitate and assemble into soluble oligomers. In general, the structure of an amyloid β peptide which assembles into oligomers is depicted by the motif R₁-(Xaa)₄-B-(Xaa)₂-₄ (SEQ ID NO:64), wherein R₁ denotes a peptide of 25 to 34 amino acid residues in length; B is a glycyl-glycyl, glycyl-prolyl, or prolyl-glycyl dipeptide located at amino acid residues 37 and 38 which is capable of forming a beta-turn; and Xaa is a natural or modified hydrophobic amino acid residue.

As is conventional in the art, a hydrophobic amino acid refers to an amino acid having a side chain that is uncharged at physiological pH and that is repelled by aqueous solution. Examples of proteinogenic hydrophobic amino acids include Ile, Leu, and Val. Examples of non-protein hydrophobic amino acids include t-BuA. Aromatic amino acids refer to hydrophobic amino acid residues having a side chain containing at least one ring having a conjugated pi-electron system (aromatic group). The aromatic group can be further substituted with substituent groups such as alkyl, alkenyl, alkynyl, hydroxyl, sulfonyl, nitro and amino groups, as well as others. Examples of proteinogenic aromatic amino acids include Phe, Tyr and Trp. Commonly encountered non-protein aromatic amino acids include Phg, 2-Nal, Thi, Phe(4-Cl), Phe(2-F), Phe(3-F) and Phe(4-F). An apolar amino acid residue refers to a hydrophobic amino acid having a side chain that is generally uncharged at physiological pH and that is not polar. Examples of proteinogenic apolar amino acids include Gly, Pro and Met. Examples of non-protein apolar amino acids include Cha. An aliphatic amino acid residue refers to an apolar amino acid having a saturated or unsaturated straight chain, branched or cyclic hydrocarbon side chain. Examples of proteinogenic aliphatic amino acids include Ala, Leu, Val and Ile. Examples of non-protein aliphatic amino acids include Nle.

More specifically, certain embodiments embrace an amyloid β peptide or amyloid β peptide analog of having the core sequence:

(SEQ ID NO:65)

R₂-Xaa₁-Gly-Ala-Ile-Ile-Gly-Leu-Xaa₂-Val-Xaa₃-
Xaa₄-Val-Val-R₃,

wherein R₂ denotes a peptide of 0 to 29 amino acid residues in length; Xaa₁ is a basic amino acid residue; Xaa₂ is methionine or a structural derivative thereof; Xaa₃ and Xaa₄ are independently glycine, D-proline or L-proline, with the proviso that both Xaa₃ and Xaa₄ are not simultaneously a proline; and R₃ is a peptide of 3 to 5 amino acid residues, 2 to 5 amino acid residues or 0 to 5 amino acid residues when the amyloid β peptide subunit is amyloid β X-43, amyloid β X-42, or amyloid β X-40, respectively.

Desirably the R₂ peptide (and likewise the R₁) is hydrophilic, i.e., overall the amino acid residues of the peptide have side chains that are attracted by aqueous solution. Moreover, it is desirable that the R₃ is hydrophobic. Exemplary R₂ and R₃ peptides are set forth herein in the amyloid β peptide subunits presented in Tables 3, 4, and 5.

Regarding Xaa₁, a basic amino acid residue refers to a hydrophilic amino acid having a side chain pK value of greater than 7. Basic amino acids typically have positively charged side chains at physiological pH due to association with hydronium ion. Examples of proteinogenic basic amino acids include arg, lys and his. Examples of non-protein basic amino acids include the non-cyclic amino acids orn, Dpr, Dbu and hArg.

Regarding Xaa₂, amino acid residues which are structural derivatives of methionine include leucine, isoleucine, valine, norvaline or norleucine. In particular embodiments, Xaa₁ is methionine, norleucine, or valine.

Advantageously, amyloid β peptides or amyloid β peptide analogs containing the core sequence set forth in SEQ ID NO:65 are capable of forming the internal beta sheet required for the formation of soluble amyloid β peptide assemblies and are therefore of use in accordance with the present invention. In this regard, the skilled artisan can appreciate that any of the above-referenced truncations, labels, additional amino acid residues, amino acid substitutions, and modified amino acid residues can be combined in any arrangement to provide an amyloid β peptide subunit for use in this invention.

As demonstrated herein, higher order assemblies of amyloid β peptide can be formed from any amyloid β peptide capable of associating with other amyloid β peptides to form soluble non-fibrillar amyloid β peptide assemblies. In certain embodiments, amyloid β 1-43 peptide monomers, amyloid β 1-42 peptide monomers, or amyloid β 1-40 peptide monomers are employed. In other embodiments, amyloid β 1-43 peptide analogs, amyloid β 1-42 peptide analogs, or amyloid β 1-40 peptide analogs are employed. In this regard, certain embodiments embrace amyloid β peptide assemblies produced from a homogenous population of amyloid β peptide subunits or monomers, e.g., amyloid β 1-43 peptides, amyloid β 1-42 peptides, amyloid β 1-40 peptides, or amyloid β peptide analogs. However, alternative embodiments embrace amyloid β peptide assemblies produced from a heterogenous population of amyloid β peptide subunits or monomers, e.g., a mixture of amyloid β 1-43 peptides with amyloid β 1-42 peptides; a mixture of amyloid β 1-43 peptides with amyloid β 1-40 peptides; a mixture of amyloid β 1-42 peptides with amyloid β 1-40 peptides; a mixture of amyloid β 1-43 peptides, amyloid β 1-42 peptides, and amyloid β 1-40 peptides; or mixtures wild-type amyloid β peptides with one or more amyloid β peptide analogs. Yet other embodiments embrace mixtures composed of one or more different amyloid β peptide analogs. In particular embodiments, heterogenic association of amyloid β peptides is achieved in the presence of at least one metal atom.

As disclosed herein, an amyloid β peptide assembly of the present invention can be identified based upon several unique features, their structure, their solubility, neurotoxic activity (e.g., in brain slices), and resistance to convert to fibrils under tissue culture conditions.

The present invention also relates to the use of metal atoms to modulate assembly distribution and stabilize neurotoxic amyloid β peptide assemblies. It has now been found that that, e.g., copper stabilizes and enriches gel-stable oligomer species. In particular, stoichiometric CuCl₂ concentrations, relative to amyloid β 1-42 monomer, greatly stabilizes the 12-mer band seen in non-copper-treated samples. The monomer band, in both cross-linked and uncross-linked samples containing copper, nearly completely converts to 12-mer species, with a small amount converting into higher integral assemblies of 12-mers, such as 24-mers, 36-mers and 48-mers. A small amount of peptide is also retained at a trimer molecular weight, indicating that soluble amyloid β assembly may proceed in a monomer ->trimer ->12-mer pathway. In addition to copper, other metals such as Mg, Zn, Fe, Co, Mn, Cr and Ni were shown to accelerate amyloid β peptide assembly. Moreover, copper was observed to stimulate assembly of the amyloid β 1-40 peptide, as well as assembly of the amyloid β 1-42 [Nle35-D-Pro37] peptide. Accordingly, particular embodiments of the present invention embrace amyloid β peptide assemblies containing or formed in the presence of a metal atom. Metal atoms of particular use in this invention include, but are not limited to Cu, Mg, Zn, Fe, Al, Co, Mn, Cr and Ni. Such metal atoms can be from any source including salts of metal atoms.

The plaque/fibril paradox inherent in the classical amyloid cascade hypothesis can be reconciled by recognizing that the relevant, proximal consequence of elevated amyloid β 1-42 is amyloid β peptide assembly, and that functional deficits emanate from synaptic disruption by these assemblies, rather than neuronal death due to plaques and fibrils. This mechanism provides a straightforward explanation for the early, subtle cognitive deficits in AD, i.e., low concentrations of amyloid β peptide assemblies trigger abnormal neuronal signaling, and the severe deficits in later-stage AD, wherein long-term exposure to increasing concentrations of amyloid β peptide assemblies leads to progressive, degenerative pathology (e.g., neurofibrillary tangles) and neuron death.

As the likely molecular cause of AD, isolated amyloid β peptide assemblies of the present invention find application as vaccines for the prevention of diseases or conditions associated with amyloid β peptide assemblies, as well as in the generation of oligomer-specific antibodies and in assays for identifying agents which modulate the formation of the assemblies, modulate binding of the assemblies to cell surface receptors, or modulate activity of the amyloid β peptide assemblies. Such agents would be particularly useful in the treatment of diseases or conditions associated with amyloid β peptide assemblies including Alzheimer's disease, Down's syndrome, mild cognitive impairment, stroke-associated dementia and the like.

In one embodiment, the soluble amyloid β peptide assemblies of the present invention are used in a vaccine. In accordance with this embodiment, the assemblies can be combined with an adjuvant to stimulate an immune response. Examples of such adjuvants include, but are not limited to, aluminum salts; Incomplete Freund's adjuvant; threonyl and n-butyl derivatives of muramyl dipeptide; lipophilic derivatives of muramyl tripeptide; monophosphoryl lipid A; 3′-de-0-acetylated monophosphoryl lipid A; cholera toxin; phosphorothionated oligodeoxynucleotides with CpG motifs; and adjuvants such as those disclosed in U.S. Pat. No. 6,558,670.

In another embodiment, any one of the isolated amyloid β peptide assemblies of the present invention can be used to generate an antibody which specifically recognizes the amyloid β peptide assembly, i.e., recognizes a linear epitope or a conformation epitope. The term antibody is used in the broadest sense and specifically includes, but is not be limited to, polyclonal or monoclonal antibodies, and chimeric, human (e.g. isolated from B cells), humanized, bispecific, neutralizing, or single chain antibodies or fragments thereof. In one embodiment, an antibody of the instant invention is monoclonal. For the production of antibodies, various hosts including goats, rabbits, chickens, rats, mice, humans, and others, can be immunized by injection of one or more of the amyloid β peptide assemblies disclosed herein either alone or in combination with an adjuvant. Methods for producing antibodies are well-known in the art. See, e.g., Kohler and Milstein ((1975) Nature 256:495-497) and Harlow and Lane (Antibodies: A Laboratory Manual (Cold Spring Harbor Laboratory, New York (1988)).

Monoclonal antibodies to amyloid β peptide assemblies can be prepared using any technique which provides for the production of antibody molecules by continuous cell lines in culture. These include, but are not limited to, the hybridoma technique, the human B-cell hybridoma technique, and the EBV-hybridoma technique (Kohler, et al. (1975) Nature 256:495-497; Kozbor, et al. (1985) J. Immunol. Methods 81:31-42; Cote, et al. (1983) Proc. Natl. Acad. Sci. 80:2026-2030; Cole, et al. (1984) Mol. Cell Biol. 62:109-120).

In addition, humanized and chimeric antibodies can be produced by splicing of mouse antibody genes to human antibody genes to obtain a molecule with appropriate antigen specificity and biological activity (see Morrison, et al. (1984) Proc. Natl. Acad. Sci. 81, 6851-6855; Neuberger, et al. (1984) Nature 312:604-608; Takeda, et al. (1985) Nature 314:452-454; Queen, et al. (1989) Proc. Natl. Acad. Sci. USA 86:10029-10033; WO 90/07861). For example, a mouse antibody is expressed as the Fv or Fab fragment in a phage selection vector. The gene for the light chain (and in a parallel experiment, the gene for the heavy chain) is exchanged for a library of human antibody genes. Phage antibodies, which still bind the antigen, are then identified. This method, commonly known as chain shuffling, provided humanized antibodies that should bind the same epitope as the mouse antibody from which it descends (Jespers, et al. (1994) Biotechnology NY 12:899-903). As an alternative, chain shuffling can be performed at the protein level (see, Figini, et al. (1994) J. Mol. Biol. 239:68-78).

Humanized antibodies can also be obtained using phage-display methods. See, e.g., WO 91/17271 and WO 92/01047. In these methods, libraries of phage are produced in which members display different antibodies on their outer surfaces. Antibodies are usually displayed as Fv or Fab fragments. Phage displaying antibodies with a desired specificity are selected by affinity enrichment to amyloid β peptide assemblies. Humanized antibodies against amyloid β peptide assemblies can also be produced from non-human transgenic mammals having transgenes encoding at least a segment of the human immunoglobulin locus and an inactivated endogenous immunoglobulin locus. See, e.g., WO 93/12227 and WO 91/10741, each incorporated herein by reference. Humanized antibodies can be selected by competitive binding experiments, or otherwise, to have the same epitope specificity as a particular mouse antibody. Such antibodies are particularly likely to share the useful functional properties of the mouse antibodies. Human polyclonal antibodies can also be provided in the form of serum from humans immunized with an immunogenic agent. Optionally, such polyclonal antibodies can be concentrated by affinity purification using amyloid β peptide assemblies as an affinity reagent.

Humanized antibodies can also be produced by veneering or resurfacing of murine antibodies. Veneering involves replacing only the surface fixed region amino acids in the mouse heavy and light variable regions with those of a homologous human antibody sequence. Replacing mouse surface amino acids with human residues in the same position from a homologous human sequence has been shown to reduce the immunogenicity of the mouse antibody while preserving its ligand binding. The replacement of exterior residues generally has little, or no, effect on the interior domains, or on the interdomain contacts. (See, e.g., U.S. Pat. No. 6,797,492).

Antibodies can be designed to have IgG, IgD, IgA, IgM or IgE constant regions, and any isotype, including IgG1, IgG2, IgG3 and IgG4. Antibodies can be expressed as tetramers containing two light and two heavy chains, as separate heavy chains and light chains or as single chain antibodies in which heavy and light chain variable domains are linked through a spacer. Techniques for the production of single chain antibodies are well-known in the art.

Diabodies are also contemplated. A diabody refers to an engineered antibody construct prepared by isolating the binding domains (both heavy and light chain) of a binding antibody, and supplying a linking moiety which joins or operably links the heavy and light chains on the same polypeptide chain thereby preserving the binding function (see, Holliger et al. (1993) Proc. Natl. Acad. Sci. USA 90:6444; Poljak (1994) Structure 2:1121-1123). This forms, in essence, a radically abbreviated antibody, having only the variable domain necessary for binding the antigen. By using a linker that is too short to allow pairing between the two domains on the same chain, the domains are forced to pair with the complementary domains of another chain and create two antigen-binding sites. These dimeric antibody fragments, or diabodies, are bivalent and bispecific. The skilled artisan will appreciate that any method to generate diabodies can be used. Suitable methods are described by Holliger, et al. (1993) supra, Poljak (1994) supra, Zhu, et al. (1996) Biotechnology 14:192-196, and U.S. Pat. No. 6,492,123, incorporated herein by reference.

Antibody fragments are also expressly encompassed by the instant invention. Fragments are intended to include Fab fragments, F(ab′)₂ fragments, F(ab′) fragments, bispecific scFv fragments, Fd fragments and fragments produced by a Fab expression library, as well as peptide aptamers. For example, F(ab′)₂ fragments are produced by pepsin digestion of the antibody molecule of the invention, whereas Fab fragments are generated by reducing the disulfide bridges of the F(ab′)₂ fragments. Alternatively, Fab expression libraries can be constructed to allow rapid and easy identification of monoclonal Fab fragments with the desired specificity (see Huse, et al. (1989) Science 254:1275-1281). In particular embodiments, antibody fragments of the present invention are fragments of neutralizing antibodies which retain the variable region binding site thereof. Exemplary are F(ab′)₂ fragments, F(ab′) fragments, and Fab fragments. See generally Immunology: Basic Processes (1985) 2^{nd} edition, J. Bellanti (Ed.) pp. 95-97.

Peptide aptamers which differentially recognize amyloid β peptide assemblies can be rationally designed or screened for in a library of aptamers (e.g., provided by Aptanomics S A, Lyon, France). In general, peptide aptamers are synthetic recognition molecules whose design is based on the structure of antibodies. Peptide aptamers consist of a variable peptide loop attached at both ends to a protein scaffold. This double structural constraint greatly increases the binding affinity of the peptide aptamer to levels comparable to that of an antibody (nanomolar range).

The invention is described in greater detail by the following non-limiting examples.

### EXAMPLE 1

### Preparation of Amyloid β Peptide Assemblies

Amyloid β peptide assemblies were prepared by dissolving 1 mg of solid amyloid β 1-42 (e.g., synthesized as described in Lambert, et al. (1994) J. Neurosci. Res. 39:377-395) in 44 μL of anhydrous DMSO. This 5 mM solution then was diluted into cold (4° C.) F12 media (GIBCO-BRL, Life Technologies) to a total volume of 2.20 mL (50-fold dilution), and vortexed for about 30 seconds. The mixture was allowed to incubate at from about 0° C. to about 8° C. for about 24 hours, followed by centrifugation at 14,000×g for about 10 minutes at about 4° C. The supernatant was diluted by factors of 1:10 to 1:10,000 into the particular defined medium, prior to incubation with brain slice cultures, cell cultures or binding protein preparations. In general, however, oligomeric assemblies were formed at a concentration of amyloid β protein of 100 μM. Typically, the highest concentration used for experiments was 10 μM and, in some cases, oligomeric assemblies (measured as initial amyloid β concentration) were diluted (e.g., in cell culture media) to 1 nM. For analysis by atomic force microscopy (AFM), a 20 μL aliquot of the 1:100 dilution was applied to the surface of a freshly cleaved mica disk and analyzed. Other manipulations were as described as follows, or as is apparent.

Alternately, assembly formation was carried out as described above, with the exception that the F12 media was replaced by a buffer (i.e., “substitute F12 media”) containing the following components: N,N-dimethylglycine (766 mg/L), D-glucose (1.802 g/L), calcium chloride (33 mg/L), copper sulfate pentahydrate (25 mg/L), iron(II) sulfate heptahydrate (0.8 mg/L), potassium chloride (223 mg/L), magnesium chloride (57 mg/L), sodium chloride (7.6 g/L), sodium bicarbonate (1.18 g/L),disodium hydrogen phosphate (142 mg/L), and zinc sulfate heptahydrate (0.9 mg/L). The pH of the buffer was adjusted to 8.0 using 0.1 M sodium hydroxide.

As yet another alternative, amyloid β protein assemblies can be prepared by pretreatment with hexafluoroisoproanol (HFIP). In accordance with this method, amyloid β monomer stock solution is made by dissolving the monomer in HFIP, which is subsequently removed by speed vacuum evaporation. The solid peptide is redissolved in dry DMSO at 5 mM to form a DMSO stock solution, and the amyloid β peptide assemblies are prepared by diluting 1 μl of the DMSO stock solution into 49 μl of F12 media (serum-free, phenol red-free). The mixture is vortexed and then incubated at 4° C. for 24 hours to generate the assemblies.

### EXAMPLE 2

### Crosslinking of Amyloid β Peptide Assemblies

Glutaraldehyde has been successfully used in a variety of biochemical systems. Glutaraldehyde tends to crosslink proteins that are directly in contact, as opposed to nonspecific reaction with high concentrations of monomeric protein. In this example, glutaraldehyde-commanded crosslinking of amyloid β peptides was investigated.

Oligomer preparation was carried out as described herein with use of substitute F12 media. The supernatant that was obtained following centrifugation (and in some cases, fractionation) was treated with 0.22 mL of a 25% aqueous solution of glutaraldehyde (Aldrich), followed by 0.67 mL of 0.175 M sodium borohydride in 0.1 M NaOH (according to the method of Levine (1995) Neurobiology of Aging 16: 755-764). The mixture was stirred at 4° C. for 15 minutes and was quenched by addition of 1.67 mL of 20% aqueous sucrose. The mixture was concentrated 5-fold on a SPEEDVAC and dialyzed to remove components smaller than 1 kD. The material was analyzed by SDS-PAGE.

Gel filtration chromatography was carried according to the following: SUPEROSE 75PC 3.2/3.0 column (Pharmacia) was equilibrated with filtered and degassed 0.15% ammonium hydrogen carbonate buffer (pH=7.8) at a flow rate of 0.02 mL/minute over the course of 18 hours at room temperature. The flow rate was changed to 0.04 mL/minute and 20 mL of solvent was eluted. Fifty μL of reaction solution was loaded on the column and the flow rate was resumed at 0.04 mL/minute. Compound elution was monitored via UV detection at 220 nm, and 0.5-1.0 mL fractions were collected during the course of the chromatography. Fraction No. 3, corresponding to the third peak of UV absorbance was isolated and demonstrated by AFM to contain globules 4.9±0.8 nm (by width analysis). This fraction was highly neurotoxic when contacted with brain slice neurons as described herein.

### EXAMPLE 3

### Size Characterization of Amyloid β Peptide Assemblies

This example sets forth the size characterization of assemblies formed as in Example 1, and using a variety of methods (e.g., native gel electophoresis, SDS-polyacrylamide gel electrophoresis, AFM, field flow fractionation, and immunorecognition).

AFM was carried out essentially according to established methods (e.g., Stine, et al. (1996) J. Protein Chem. 15:193-203, 1996). Namely, images were obtained using a Digital Instruments (Santa Barbara, Calif.) Nanoscope IIIa Multimode Atomic force microscope using a J-scanner with xy range of 150μ. Tapping Mode was employed for all images using etched silicon TESP Nanoprobes (Digital Instruments). AFM data was analyzed using the Nanoscope IIIa software and the IGOR Pro™ waveform analysis software. For AFM analysis, 4μ scans (i.e., assessment of a 4 μm×4 μm square) were conducted. Typically, dimensions reported were obtained by section analysis, and where width analysis was employed, it is specified. Section and width analysis are in separate analysis modules in the Nanoscope IIIa software. Generally, for ADDL analysis, there is a systematic deviation between the sizes obtained by section analysis and those obtained by width analysis. Namely, for a 4μ scan, section analysis yielded heights that were usually about 0.5 nm taller, thus resulting in a deviation of about 0.5 nm in the values obtained for the sizes of the globules.

Analysis by gel electrophoresis was carried out on 15% polyacrylamide gels and visualized by COOMASSIE blue staining. Assemblies were resolved on 4-20% Tris-glycine gels under non-denaturing conditions (Novex). Electrophoresis was performed at 20 n-LA for approximately 1.5 hours. Proteins were resolved with SDS-PAGE as described in Zhang, et al. ((1994) J. Biol. Chem. 269:25247-25250). Protein was then visualized using silver stain (e.g., as described in Sherchenko, et al. (1996) Anal. Chem. 68:850-858). Gel proteins from both native and SDS gels were transferred to nitrocellulose membranes according to Zhang, et al. ((1994) supra). Immunoblots were performed with biotinylated 6E10 antibody (Senetak, Inc., St. Louis, Mo.) at 1:5000 and visualized using ECL (Amersham). Typically, gels were scanned using a densitometer. This allowed provision of the computer-generated images of the gels (e.g., versus photographs of the gels themselves).

Size characterization of assemblies by AFM section analysis (e.g., as described in Stine, et al. (1996) J. Protein Chem. 15:193-203) or width analysis (Nanoscope III software) indicated that the predominant species were globules of about 4.7 nm to about 6.2 nm along the z-axis. Comparison with small globular proteins (amyloid β 1-40 monomer, aprotinin, bFGF, carbonic anhydrase) suggested that amyloid β peptide assemblies had masses between 17-54 kD. Distinct species were recognized. Globules of dimensions of from about 4.9 nm to about 5.4 nm, from about 5.4 nm to about 5.7 nm, and from about 5.7 nm to about 6.2 nm were apparent. The globules of dimensions of about 4.9-5.4 nm and 5.7-6.2 nm appeared to constitute about 50% of the oligomeric structures in a typical sample. There also appeared to be a distinct size species of globule having dimensions of from about 5.3 nm to about 5.7 nm. The globules of dimensions of from about 4.7 nm to about 6.2 nm on AFM were deemed the hexamer and dodecamer form of oligomeric amyloid β peptide; whereas the globules of from about 4.2 nm to about 4.7 nm appeared to correspond to the amyloid β tetramer; and the size globules of from about 3.4 nm to about 4.0 nm appeared to correspond to trimer. In contrast, the size globules of from about 2.8 nm to about 3.4 nm corresponded to dimer (Roher, et al. (1996) J. Biol. Chem. 271:20631-20635) and the amyloid β monomer AFM size ranges were from about 0.8 nm to about 1.8-2.0 nm.

In agreement with the AFM analysis, SDS-PAGE immunoblots of amyloid β peptide assemblies identified amyloid β oligomers of about 17 kD to about 22 kD, with abundant 4 kD monomer present, presumably a breakdown product. Consistent with this interpretation, non-denaturing polyacrylamide gels of ADDLs showed scant monomer, with a primary band near 30 kD, a less abundant band at 17 kD, and no evidence of fibrils or aggregates. The correspondence between the SDS and non-denaturing gels confirmed that the small oligomeric size of amyloid β peptide assemblies was not due to detergent action. Oligomers seen in ADDL preparations were smaller than clusterin (Mr 80 kD, 40 kD in denatured gels), as expected from use of low clusterin concentrations (1/40 relative to amyloid β, which precluded association of amyloid β as 1:1 amyloid β-clusterin complexes).

An amyloid β peptide assembly preparation according to the invention was fractionated on a SUPERDEX 75 column (Pharmacia, SUPEROSE 75PC 3.2/3.0 column). The fraction containing the ADDLs was the third fraction of UV absorbance eluting from the column and was analyzed by AFM and SDS-polyacryalamide gel electrophoresis. Fractionation resulted in greater homogeneity for the ADDLs, with the majority of the globules having dimensions of from about 4.9 nm to about 5.4 nm. SDS-PAGE of the fraction demonstrated a heavy lower band corresponding to the monomer/dimer form of amyloid β. As also observed for the non-fractionated preparation of ADDLs, this appeared to be a breakdown product of the amyloid β peptide assemblies. Heavier loading of the fraction revealed a larger-size broad band (perhaps a doublet). This further confirmed the stability of the non-fibrillar oligomeric amyloid β peptide structures to SDS.

To detect even higher order oligomers (i.e., >13 mers), 1 μl of the oligomer solution, prepared as in Example 1, was added to four μl of F12 and 5 μl of Tris-tricine loading buffer, and then loaded on a pre-made 16.5% Tris-tricine gel (BIO-RAD). Electrophoresis was carried out for 2.25 hours at 100 V. Following electrophoresis, the gel was stained using the Silver Xpress kit (Novex). Alternately, instead of staining the gel, the amyloid β species were transferred from the gel to HYBOND-ECL (Amersham) in SDS-containing transfer buffer for 1 hour at 100 V at 4° C. The blot was blocked in TBS-T1 containing 5% milk for 1 hour at room temperature. Following washing in TBS-T1, the blot was incubated with primary antibody (26D6, 1:2000) for 1.5 hours at room temperature. The 26D6 antibody recognizes the amino terminal region of amyloid β. Following further washing, the blot was incubated with secondary antibody (anti-mouse HRP, 1:3500) for 1.5 hours at room temperature. Following more washing, the blot was incubated in WEST PICO SUPERSIGNAL reagents (500 μl of each, supplied by Pierce) and 3 mls of ddH₂0 for 5 minutes. Finally, the blot was exposed to film and developed.

Results of this analysis confirmed a range of soluble amyloid β peptide assemblies, dimer amyloid β, and monomer amyloid β. This gel system thus enables visualization of distinct amyloid p peptide assemblies of from at least three monomers (trimer) up to about 24 monomers.

AFM analysis was also carried out on these higher order oligomers except that fractionation on a SUPERDEX 75 column was not performed, and the field was specifically selected such that larger size globules in the field were measured. AFM was carried out using a NANOSCOPE2 III MultiMode AFM (MMAFM) workstation using TAPPINGMODE2 (Digital Instruments, Santa Barbara, Calif.). The results of these studies showed structures ranging in size from 1 to 10.5 nm in z height. Based on this characterization, the assemblies were composed of 3 to 24 monomeric subunits, consistent with the bands shown on Tris-tricine SDS-PAGE. In separate experiments, species as high as about 11 nm were also observed.

### EXAMPLE 4

### Clusterin Treatment of Amyloid β

Although it has been proposed that fibrillar structures represent the toxic form of amyloid β (Lorenzo, et al. (1994) Proc. Natl. Acad. Sci. USA, 91:12243-12247; Howlett, et al. (1995) Neurodegen. 4:23-32), novel neurotoxins that do not behave as sedimentable fibrils will form when amyloid β 1-42 is incubated with low doses of clusterin, which also is known as “Apo J” (Oda, et al. (1995) Exper. Neurol. 136:22-31; Oda, et al. (1994) Biochem. Biophys. Res. Commun. 204:1131-1136). To determine whether these slowly sedimenting toxins might contain small or nascent fibrils, clusterin-treated amyloid β preparations were examined by atomic force microscopy.

Clusterin treatment was carried out as described in Oda, et al. (1995) supra) basically by adding clusterin in the incubation as described in Example 1. Alternatively, the starting amyloid β 1-42 could be dissolved in 0.1 N HCl, rather than DMSO, and this starting amyloid β 1-42 could even have fibrillar structures at the outset. However, incubation with clusterin for 24 hours at room temperature of 37° C. resulted in preparations that were predominantly free of fibrils, consistent with their slow sedimentation. This was confirmed by experiments showing that fibril formation decreased as the amount of clusterin added increased.

The preparations resulting from clusterin treatment were exclusively composed of small globular structures approximately 5-6 nm in size as determined by AFM analysis of ADDLs fractionated on a SUPERDEX 75 gel column. Equivalent results were obtained by conventional electron microscopy. In contrast, amyloid β 1-42 that had self-associated under standard conditions (Snyder, et al. (1994) Biophys. J. 67:1216-28, 1994) in the absence of clusterin showed primarily large, non-diffusible fibrillar species. Moreover, the resultant ADDL preparations were passed through a CENTRICON 10 kD cut-off membrane and analyzed on as SDS-polyacrylamide gradient gel. The results of this analysis indicated that only the monomer passed through the CENTRICON 10 filter, whereas ADDLs were retained by the filter. Monomer found after the separation could only be formed from the larger molecular weight species retained by the filter.

These results confirm that toxic amyloid β peptide assembly preparations are composed of small fibril-free oligomers of amyloid β peptide, and that ADDLs can be obtained by appropriate clusterin treatment of amyloid β.

### EXAMPLE 5

### Physiologic Formation of Amyloid β Peptide Assemblies

The toxic moieties in Example 4 could be composed of rare structures that contain oligomeric amyloid β and clusterin. Whereas Oda, et al. ((1995) supra) reported that clusterin was found to increase the toxicity of amyloid β 1-42 solutions, others have found that clusterin at stoichiometric levels protects against amyloid β 1-40 toxicity (Boggs, et al. (1997) J. Neurochem. 67:1324-1327, 1997). Accordingly, ADDL formation in the absence of clusterin was further characterized. The results of this analysis indicated that amyloid β self-associated in low temperature solutions, forming amyloid β peptide assemblies essentially indistinguishable from those chaperoned by clusterin.

### EXAMPLE 6

### Biotin-Labeled Amyloid β Peptide Assemblies

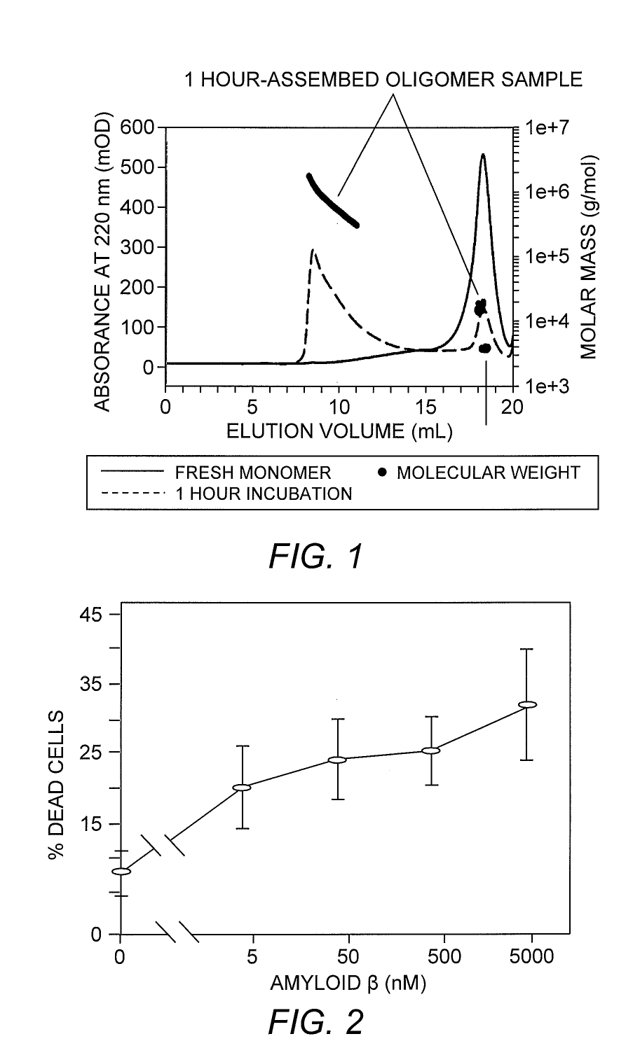

The incorporation of biotin into amyloid β peptide assemblies allows for the direct detection of amyloid β peptide assemblies using streptavidin-linked reagents. In this regard, biotin-amyloid β peptide assemblies were produced and found to oligomerize into trimer/tetramer and HMW assemblies. When used in a 1:4 ratio with native amyloid β 1-42, biotin-amyloid 1 1-42 allowed for the correct profile of ADDL assembly. A one hour incubation of 100 μM total peptide (20 amyloid μM biotinylated amyloid β 1-42, 80 μM wild-type amyloid β 1-42) in 1×PBS (without Ca and Mg) at 37° C. lead to significant soluble oligomer formation, compared to the fresh peptide monomer dilution at time zero. One ml samples were produced using standard amyloid β peptide assembly preparation methodologies, but using PBS as diluent, after HFIP evaporation and DMSO resuspension, instead of F-12 tissue culture medium. Oligomerization curves were obtained by monitoring the absorbance (200 nm) of 300 μl samples injected onto a L SUPERDEX-200 HR 10/30 column at a flow rate of 0.5 ml/minute in 1×PBS (without Ca and Mg) at room temperature. An AKTA Basic chromatography system, using Unicorn software, operated the system and collected the data. The dashed line in FIG. 1 represents the molecular weight (MW) values determined by Multi-Angle Laser Light Scattering (MALLS). A Wyatt Technologies DAWN EOS MALLS instrument was connected inline with the HPLC column and absorbance flow cell, and an Optilab rEX instrument was used to determine the protein concentration of eluting species. Using Wyatt Technologies' ASTRA V software, the MW profiles were recorded and fitted. As can be seen in the time zero fresh monomer sample, a MW corresponding to monomer was observed in the second peak, eluting at roughly 19 minutes (see FIG. 1). The one hour sample (dashed line) had significantly oligomerized. The first peak, which trailed significantly from 8 ml to 15 ml, contained species from the million Dalton range to the low hundred thousands of Dalton. The second peak, rather than containing predominantly monomeric peptide, now contains species in the trimer and tetramer range. While the monomer MW was roughly 4800 Da, 1 hour sample contained low molecular weight (LMW) oligomers in the 15000 to 20000 range, indicating stable formation of these species. Fluorescein labeled amyloid β peptides assembled similarly to biotin labeled amyloid β peptides.

### EXAMPLE 7

### Characterization of Biotin-Labeled Amyloid β Peptide Assemblies

Amyloid β peptide assemblies were prepared from a mixture (1:4.7 mol:mol) of biotinylated and unlabeled amyloid β 1-42 by mixing HFIP solutions of the two peptides and air drying overnight followed by drying on a SAVANT SPEED-VAC dryer. The HFEP film was dissolved in DMSO to ˜5 mM and diluted with ice cold F12 to ˜100 μM, vortexed briefly and allowed to incubate at 4° C. overnight. The sample was centrifuged at 14,000×g for 10 minutes at 4° C. and transferred to a clean tube. Protein concentration was determined by COOMASSIE Plus protein assay (Pierce) using a BSA standard. Biotinylated amyloid β peptide assemblies were subjected to size exclusion chromatography (SEC) on a SUPERDEX 75 HR/10/30 column and the fractions analyzed by dot blot for distribution of the biotin label. Biotinylated amyloid β peptide assemblies and SEC fractions were diluted with F-12 and native sample buffer (final concentration of 5 mM Tris-HCl, pH 6.8, 38.3 mM glycine, 10% glycerol, 0.017% bromphenol blue) or Tricine sample buffer (BIO-RAD) and analyzed (˜60 pmoles for silver stain or ˜20 pmoles for western blot analysis) by PAGE. Unlabeled amyloid β peptide assemblies were run for comparison. The native gel (10% T acrylamide, 5% C resolving gel) used a running buffer of 5 mM Tris, 38.4 mM glycine, pH 8.3 (Berts, et al. (1999) Meth. Enzymol. 309:333-350) at 120 V, 4° C. for ˜3 hours. The SDS gel (10-20% Tris-Tricine precast gel, BIO-RAD) was run with Tris/glycine/SDS buffer (BIO-RAD) at 120 V for 80 minutes at room temperature. Silver stain was performed with a SILVERXPRESS silver stain kit (INVITROGEN) using the Tricine gel protocol. Alternatively, the gels were electroblotted onto HYBOND ECL nitrocellulose using 25 mM Tris-192 mM glycine, 20% v/v methanol, pH 8.3 at 100V for 1 hour at 4° C. The blots were blocked with 5% milk in TBS-T (0.1% TWEEN-20 in 20 mM Tris-HCl, pH 7.5, 0.8% NaCl) for 1 hour at room temperature.

Biotin Probe. An avidin-biotinylated HRP complex (VECTASTAIN ABC standard kit; Vector Labs) was formed by diluting the A and B reagents 1:500 in 5% milk/TBS-T and pre-incubating for 30 minutes at room temperature. The blots were incubated with the preformed complex for 1 hour and washed three times, 10 minutes each, with TBS-T, rinsed two times with dH₂0, developed with SUPERSIGNAL West Femto Maximum Sensitivity substrate (Pierce; 1:1 dilution with ddH₂O) and read on a KODAK Image Station.

Immunostain. Monoclonal anti-amyloid β peptide (6E10, Signet) or anti-amyloid β peptide assembly (20C2, 1.52 mg/ml) were diluted 1:1000 in milk/TBS and incubated with the blots for 90 minutes at room temperature. Following washing three times, 10 minutes each, with TBS-T, the blots r were incubated with HRP-linked anti-mouse Ig (1:40,000 in milk/TBST; Amersham) for 90 minutes at room temperature. The blots were washed three times, 10 minutes each, with TBS-T, rinsed two times with dH₂O, developed with SUPERSIGNAL West Femto Maximum Sensitivity substrate (Pierce; 1:1 dilution with ddH₂O) and read on a KQDAK Image Station.

The results of this analysis indicated that biotinylated amyloid β peptide assemblies have a SEC profile similar to that previously observed using unlabeled amyloid β peptide assemblies. The dot blot for the biotin label showed a similar profile to the absorbance readings at 280 nm. The native-PAGE western blot of SEC fractions using a probe for the biotin label showed slower moving oligomers in Peak 1. Most of the major native species, as well as a faster moving band, were in Peak 2. There was no staining in Peak 3 fractions. Silver stain of biotinylated amyloid β peptide assemblies following SDS-PAGE showed a similar pattern to unlabeled amyloid β peptide assemblies. There was a single minor band at ˜52 kDa in the biotinylated amyloid β peptide assemblies. Western blot following SDS-PAGE of biotinylated and unlabeled amyloid β peptide assemblies showed specificity of the probe for biotin. Both 6E10 and 20C2 showed similar immunostaining patterns for biotinylated and unlabeled amyloid β peptide assemblies. The ˜52 kDa band in silver stain did not appear in any of the western blots. This analysis indicated that the mixture of biotinylated and unlabeled amyloid β 1-42 forms amyloid β peptide assemblies with typical electrophoretic profiles on both native and SDS gels. By probing for the biotin label, distribution of the various oligomeric species could be detected independent of the epitope-specific immunostaining obtained with antibodies. Biotinylated amyloid β peptide assemblies also fractionate on size exclusion chromatography in a similar pattern as unlabeled amyloid β peptide assemblies.

### EXAMPLE 8

### Assemblies are Diffusible, Extremely Potent CNS Neurotoxins

Whether amyloid β peptide assemblies were induced by clusterin, low temperature, or low amyloid β concentration, the stable oligomers that formed were potent neurotoxins. Toxicity was examined in organotypic mouse brain slice cultures, which provided a physiologically relevant model for mature CNS. Brain tissue was supported at the atmosphere-medium interface by a filter in order to maintain high viability in controls.

For these experiments, brain slices were obtained from strains B6 129 F2 and JR 2385 (Jackson Laboratories) and cultured using established methods (Stoppini, et al. (1991) J. Neurosci. Meth. 37:173-182), with modifications. Namely, an adult mouse was sacrificed by carbon dioxide inhalation, followed by rapid decapitation. The head was immersed in cold, sterile dissection buffer (94 mL Gey's balanced salt solution, pH 7.2, supplemented with 2 mL 0.5M MgCl₂, 2 ml 25% glucose, and 2 mL 1.0 M HEPES), after which the brain was removed and placed on a sterile Sylgard-coated plate. The cerebellum was removed and a mid-line cut was made to separate the cerebral hemispheres. Each hemisphere was sliced separately. The hemisphere was placed with the mid-line cut down and a 30 degree slice from the dorsal side was made to orient the hemisphere. The hemisphere was glued cut side down on the plastic stage of a Campden tissue chopper (previously wiped with ethanol) and immersed in ice cold sterile buffer. Slices of 200 μm thickness were made from a lateral to medial direction, collecting those in which the hippocampus was visible.

Each slice was transferred with the top end of a sterile pipette to a small petri dish containing Dulbecco's Modified Eagle Medium (DMEM) containing 10% fetal calf serum, 2% S/P/F (streptomycin, penicillin, and fungizone; Life Technologies (GIBCO-BRL), Gaithersburg, Md.), observed with a microscope to verify the presence of the hippocampus, and placed on a MILLICELL-CM insert (MILLIPORE) in a deep well tissue culture dish (FALCON, 6-well dish). Each well contained 1.0 mL of growth medium, and usually two slices were on each insert. Slices were placed in an incubator (6% CO₂, 100% humidity) overnight. Growth medium was removed and wells were washed with 1.0 mL warm Hanks BSS (GIBCO-BRL, Life Technologies). Defined medium (DMEM, N2 supplements, SPF, e.g., as described in Bottenstein, et al. (1979) Proc. Natl. Acad. Sci. USA 76:514-517) containing the amyloid β oligomers, with or without inhibitor compounds, was added to each well and the incubation was continued for 24 hours.

Cell death was measured using the LIVE/DEAD® assay kit (Molecular Probes, Eugene, Oreg.). This a dual-label fluorescence assay in which live cells are detected by the presence of an esterase that cleaves calcein-AM to calcein, resulting in a green fluorescence. Dead cells take up ethidium homodimer, which intercalates with DNA and has a red fluorescence. The assay was carried out according to the manufacturer's directions at 2 μM ethidium homodimer and 4 μM calcein. Images were obtained within 30 minutes using a NIKON DIAPHOT microscope equipped with epifluorescence. The METAMORPH image analysis system (Universal Imaging Corporation, Philadelphia, Pa.) was used to quantify the number and/or area of cells showing green or red fluorescence.

For these experiments, ADDLs were present for 24 hours at a maximal 5 μM dose of total amyloid β (i.e. , total amyloid β was never more than 5 μM in any ADDL experiment). Cell death, as shown by “false yellow staining”, was almost completely confined to the stratum pyramidale (CA 3-4) and dentate gyrus (DG) indicating that principal neurons of the hippocampus (pyramidal and granule cells, respectively) are the targets of ADDL-induced toxicity. Furthermore, glia viability was unaffected by a 24-hour ADDL treatment of primary rat brain glia, as determined by trypan blue exclusion and MTT assay. Dentate gyrus (DG) and CA3 regions were particularly sensitive and showed ADDL-evoked cell death in every culture obtained from animals aged P20 (weanlings) to P84 (young adult). Up to 40% of the cells in this region died following chronic exposure to ADDLs. The pattern of neuronal death was not identical to that observed for NMDA, which killed neurons in DG and CA1 but spared CA3.

Some cultures from hippocampal DG and CA3 regions of animals more than 20 days of age were treated with conventional preparations of fibrillar amyloid β. Consistent with the non-diffusible nature of the fibrils, no cell death (yellow staining) was evident even at 20 μM. The staining pattern for live cells in this culture verified that the CA3/dentate gyrus region of the hippocampus was being examined. The extent of cell death observed after conventional amyloid β treatment (i.e., fibrillar amyloid β preparations) was indistinguishable from negative controls in which cultures were given medium, or medium with clusterin supplement. In typical controls, cell death was less than 5%. In fact, high viability in controls could be found even in cultures maintained several days beyond a typical experiment, which confirms that cell survival was not compromised by standard culture conditions.

A dose-response experiment was carried out to determine the potency of amyloid β peptide assemblies in evoking cell death. Image analysis was used to quantify dead cell and live cell staining in fields containing the DG/CA3 areas. FIG. 2 illustrates the % dead cells verses ADDL concentration measured as initial amyloid β 1-42 concentration (nM). Because of the difficulties of quantifying brain slices, the results were not detailed enough to determine the EC50 with precision. However, as can be seen in FIG. 2, even after 1000-fold dilution (˜5 nM amyloid β), ADDL-evoked cell death was more than 20%. Toxicity was observed even with 0.3 nM ADDLs. This contrasts with results obtained with conventionally aged amyloid β, which is toxic to neurons in culture at about 20 to about 50 μM. These data demonstrate that amyloid β peptide assemblies are effective at doses approximately 1,000 to 10,000-fold lower than those used in fibrillar amyloid β experiments.

These data from hippocampal slices thus confirm the ultratoxic nature of amyloid β peptide assemblies. Furthermore, because the amyloid β peptide assemblies had to pass through the culture-support filter to cause cell death, the results validate that amyloid β peptide assemblies are diffusible, consistent with their small oligomeric size. Also, the methods set forth herein can be employed as an assay for ADDL-mediated changes in cell viability. In particular, the assay can be carried out by coincubating or coadministering ADDLs with agents that potentially may increase or decrease ADDL formation and/or activity. Results obtained with such coincubation or coadministration can be compared to results obtained with analysis of ADDLs alone.

### EXAMPLE 9

### MTT Oxidative Stress Toxicity Assays

This example sets forth an assay that can be employed to detect an early toxicity change in response to amyloid β oligomers. For these experiments, PC12 cells were passaged at 4×10⁴ cells/well on a 96-well culture plate and grown for 24 hours in DMEM +10% fetal calf serum +1% S/P/F (streptomycin, penicillin, and fungizone). Plates were treated with 200 μg/mL poly-L-lysine for 2 hours prior to cell plating to enhance cell adhesion. One set of six wells was left untreated and fed with fresh media, while another set of wells was treated with the vehicle control (PBS containing 10% 0.01 N HCl, aged overnight at room temperature). Positive controls were treated with TRITON (1%) and sodium azide (0.1%) in normal growth media. Amyloid β oligomers prepared as described in Example 1, or obtained upon coincubation with clusterin, with and without inhibitor compounds present, were added to the cells for 24 hours. After the 24 hour incubation, MTT (0.5 mg/mL) was added to the cells for 2.5 hours (11 μL of 5 mg/ml stock solubilized in PBS into 100 μL of media). Healthy cells reduce the MTT into a formazan blue colored product. After the incubation with MTT, the media was aspirated and 100 μL of 100% DMSO was added to lyse the cells and dissolve the blue crystals. The plate was incubated for 15 minutes at room temperature and read on a plate reader (ELISA) at 550 nm.

The results of this experiment indicated that control cells not exposed to ADDLs, cells exposed to clusterin alone, and cells exposed to monomeric amyloid β showed no cell toxicity. By contrast, cells exposed to amyloid β assembled with clusterin and aged one day showed a decrease in MTT reduction, evidencing an early toxicity change. The lattermost amyloid preparations were confirmed by AFM to lack amyloid fibrils. Results of this experiment thus confirm that ADDL preparations obtained from assembly of amyloid β mediated by clusterin have enhanced toxicity. Moreover, the results confirm that the PC12 oxidative stress response can be employed as an assay to detect early cell changes due to ADDLs.

In an alternative MTT oxidative stress toxicity assay, HN2 cells are used instead of PC12 cells. For this assay, HN2 cells are passaged at 4×10⁴ cells/well on a 96-well culture plate and grown for 24 hours in DMEM +10% fetal calf serum +1% S/P/F (streptomycin, penicillin, and fungizone). Plates are treated with 200 μg/mL poly L-lysine for 2 hours prior to cell plating to enhance cell adhesion. The cells are differentiated for 24 to 48 hours with 5 μM retinoic acid and growth is further inhibited with 1% serum. One set of wells is left untreated and given fresh medium. Another set of wells is treated with the vehicle control (0.2% DMSO). Positive controls are treated with TRITON (1%) and sodium azide (0.1%). Amyloid β oligomers prepared as described in Example 1, with and without inhibitor compounds present, are added to the cells for 24 hours. After the 24-hour incubation, MTT (0.5 mg/mL) is added to the cells for 2.5 hours (11 μL of 5 mg/mL stock into 100 μL of media). After the incubation with MTT, the media is aspirated and 100 μL of 100% DMSO is added to lyse the cells and dissolve the blue crystals. The plate is incubated for 15 minutes at room temperature and read on a plate reader (ELISA) at 550 nm.

It is contemplated that these assays can be used to identify the presence of amyloid β peptide assemblies as well as used to identify agents that increase or decrease ADDL formation and/or activity.

### EXAMPLE 10

### Cell Morphology by Phase Microscopy

This example sets forth yet another assay of ADDL-mediated cell changes—assay of cell morphology by phase microscopy. For this assay, cultures were grown to low density (50-60% confluence). To initiate the experiment, the cells were serum-starved in F12 media for 1 hour. Cells were then incubated for 3 hours with amyloid β oligomers prepared as described in Example 1, with and without inhibitor compounds added to the cells, for 24 hours. After 3 hours, cells were examined for morphological differences or fixed for immunofluorescence labeling. Samples were examined using the METAMORPH Image Analysis system and an MRI video camera (Universal Imaging, Inc.).

### EXAMPLE 11

### FACScan Assay for Cell Surface Binding

Because cell surface receptors recently have been identified on glial cells for conventionally prepared amyloid β (Yan, et al. (1996) Nature 382:685-691; El Khoury, et al. (1996) Nature 382:716-719), and because neuronal death at low ADDL doses suggested possible involvement of signaling mechanisms, experiments were undertaken to determine if specific cell surface binding sites on neurons exist for ADDLs.

For flow cytometry, cells were dissociated with 0.1% trypsin and plated at least overnight onto tissue culture plastic at low density. Cells were removed with cold phosphate-buffered saline (PBS)/0.5 mM EDTA, washed three times and resuspended in ice-cold PBS to a final concentration of 500,000 cells/mL. Cells were incubated in cold PBS with amyloid β peptide assemblies prepared as described in Example 1, except that 10% of the amyloid y was an amyloid β 1-42 analog containing biocytin at position 1 replacing aspartate. Oligomers with and without inhibitor compounds present were added to the cells for 24 hours. The cells were washed twice in cold PBS to remove free, unbound amyloid β oligomers, resuspended in a 1:1,000 dilution of avidin conjugated to fluorescein, and incubated for one hour at 4° C. with gentle agitation. Alternately, amyloid β-specific antibodies and fluorescent secondary antibody were employed instead of avidin, eliminating the need to incorporate 10% of the biotinylated amyloid β analog. Namely, biotinylated 6E10 monoclonal antibody (1 μL Senetec, Inc., St. Louis, Mo.) was added to the cell suspension and incubated for 30 minutes. Bound antibody was detected after pelleting cells and resuspending in 500 μL PBS, using FITC-conjugated streptavidin (1:500, Jackson Laboratories) for 30 minutes.

Cells were analyzed by a Becton-Dickenson Fluorescence Activated Cell Scanner (FACScan). Ten thousand or 20,000 events typically were collected for both forward scatter (size) and fluorescence intensity, and the data were analyzed by CONSORT 30 software (Becton-Dickinson). Binding was quantified by multiplying mean fluorescence by total number of events, and subtracting value for background cell fluorescence in the presence of 6E10 and FITC.

For these experiments, FACScan analysis was conducted to compare ADDL immunoreactivity in suspensions of log-phase yeast cells (a largely carbohydrate surface) with the B103 CNS neuronal cell line (Schubert, et al. (1974) Nature 249:224-227). For B103 cells, addition of ADDLs caused a major increase in cell associated fluorescence. Trypsin treatment of the B103 cells for 1 minute eliminated ADDL binding. In contrast, control yeast cells demonstrated no ADDL binding, verifying the selectivity of amyloid β peptide assemblies for proteins present on the cell surface. Suspensions of hippocampal cells (trypsinized tissue followed by a two-hour metabolic recovery) also bound amyloid β peptide assemblies, but with a reduced number of binding events compared with the B103 cells, as indicated by the reduced fluorescence intensity of the labeled peak.

These results thus indicate that the amyloid β peptide assemblies exert their effects by binding to a specific cell surface receptor. In particular, the trypsin sensitivity of B103 cells showed that their ADDL binding sites were cell surface proteins and that binding was selective for a subset of particular domains within these proteins.

Given the results provided herein, this assay can also be employed in screening assays to identify agents that increase or decrease ADDL formation and/or activity.

### EXAMPLE 12

### Inhibition of Cell Surface Binding

Because B103 cell trypsinization was found to block subsequent ADDL binding, experiments were performed to determine whether tryptic fragments released from the cell surface retard ADDL binding activity. Tryptic peptides were prepared using confluent B103 cells from four 100 mm dishes that were removed by trypsinization (0.025%, Life Technologies) for approximately 3 minutes. Trypsin-chymotrypsin inhibitor (0.5 mg/mL in Hank's Buffered Saline; Sigma, St. Louis, Mo.) was added, and cells were removed via centrifugation at 500×g for 5 minutes. Supernatant (˜12 mL) was concentrated to approximately 1.0 mL using a CENTRICON 3 filter (AMICON), and was frozen after the protein concentration was determined. For blocking experiments, sterile concentrated tryptic peptides (0.25 mg/mL) were added to organotypic brain slices or to suspended B103 cells along with amyloid β peptide assemblies.

In FACScan assays, tryptic peptides released into the culture media (0.25 mg/mL) inhibited ADDL binding by >90%. By comparison, control cells exposed to BSA, even at 100 mg/mL, had no loss of binding. Tryptic peptides, if added after amyloid β peptide assemblies were already attached to cells, did not significantly lower fluorescence intensities. This indicated that the peptides did not compromise the ability of the assay to quantify bound amyloid β peptide assemblies. Besides blocking ADDL binding, the tryptic peptides also were antagonists of ADDL-evoked cell death. In this regard, addition of tryptic peptides resulted in a 75% reduction in cell death, p<0.002.

These data confirm that particular cell surface proteins mediate amyloid β peptide assembly binding, and that solubilized tryptic peptides from the cell surface provide neuroprotective, ADDL-neutralizing activity.

### EXAMPLE 13

### Dose Response Curve of Cell Binding

This example sets forth dose response experiments to determine whether ADDL binding to the cell surface is saturable. For these studies, B103 cells were incubated with increasing amounts of amyloid β peptide assemblies and binding was quantitated by FACscan analysis. These results confirm that a distinct plateau is achieved for ADDL binding. Saturability of ADDL binding occurred at a relative amyloid β 1-42 concentration (i.e., amyloid β peptide assembly concentration relative to amyloid ) of about 250 nm, thereby confirming that ADDL binding is saturable. Such saturability of ADDL binding, especially when considered with the results of the trypsin studies, indicates that the amyloid β peptide assemblies are acting through a particular cell surface receptor.

### EXAMPLE 14

### Cell-Based ELISA for Binding Activity

This example sets forth a cell-based assay, particularly a cell-based enzyme-linked immunosorbent assay (ELISA) that can be employed to assess amyloid β peptide assemblies binding activity. For these studies, 48 hours prior to conduct of the experiment, 2.5×10⁴ B103 cells present as a suspension in 100 μL DMEM were placed in each assay well of a 96-well microtiter plate and kept in an incubator at 37° C. Twenty-four hours prior to conducting the experiment, amyloid β peptide assemblies were prepared according to the method described in Example 1. To begin the assay, each microtiter plate well containing cells was treated with 50 μL of fixative (3.7% formalin in DMEM) for 10 minutes at room temperature. This fixative/DMEM solution was removed and a second treatment with 50 μL formalin (no DMEM) was carried out for 15 minutes at room temperature. The fixative was removed and each well was washed twice with 100 μL PBS. Two hundred μL of a blocking agent (1% BSA in PBS) was added to each well and incubated at room temperature for 1 hour. After two washes with 100 μL PBS, 50 μL of ADDLs (previously diluted 1:10 in PBS), were added to the appropriate wells, or PBS alone as a control, and the resulting wells were incubated at 37° C. for 1 hour. Three washes with 100 μL PBS were carried out, and 50 μL biotinylated 6E10 (Senetek) diluted 1:1000 in 1% BSA/PBS was added to the appropriate wells. In other wells, PBS was added as a control. After incubation for 1 hour at room temperature on a rotator, the wells were washed three times with 50 μL PBS, and 50 μL of the ABC reagent (ELITE ABC kit, Vector Labs) was added and incubated for 30 minutes at room temperature on the rotator. After washing four times with 50 μL PBS, 50 μL of ABTS substrate solution was added to each well and the plate was incubated in the dark at room temperature. The plate was analyzed for increasing absorption at 405 nm. Only when amyloid β peptide assemblies, cells, and 6E10 were present was there a significant signal.

These results further demonstrate the utility of a cell-based ELISA assay for monitoring ADDL-mediated cell binding. It is contemplated that this assay can be used in the identification of agents that increase or decrease ADDL formation and/or activity.

### EXAMPLE 15

### Protection Against ADDL Neurotoxicity

To determine the signal transduction pathways involved in ADDL toxicity, brain slices from isogenic fyn^{-/-} and fyn^{+/+} animals were incubated with amyloid β peptide assemblies. Fyn belongs to the Src-family of protein tyrosine kinases, which are central to multiple cellular signals and responses (Clarke, et al. (1995) Science 268:233-238). Fyn is of particular interest because it is upregulated in AD-afflicted neurons (Shirazi, et al. (1993) Neuroreport 4:435-437, 1993). It also appears to be activated by conventional amyloid β preparations (Zhang, et al. (1996) Neurosci. Letts. 211:187-190). Fyn knockout mice, moreover, have reduced apoptosis in the developing hippocampus (Grant, et al. (1992) Science 258:1903-1910).

For these studies, brain slice cell cultures of Fyn knockout mice (Grant, et al. (1992) supra) were treated with amyloid β peptide assemblies. By comparing images of brain slices of mice either treated or not treated with ADDLs for 24 hours it was found that, in contrast to cultures from wild-type animals, cultures from fyn^{-/-} animals showed negligible ADDL-evoked cell death. For amyloid β peptide assemblies, the level of cell death in fyn^{+/+} slices was more than five times that in fyn^{-/-} cultures. In fyn^{-/-} cultures, cell death in the presence of ADDLs was at background level. The neuroprotective response was selective; hippocampal cell death evoked by NMDA receptor agonists (Bruce, et al. (1995) Exper. Neurol. 132:209-219; Vornov, et al. (1991) Neurochem. 56:996-1006) was unaffected. Analysis (ANOVA) using the Tukey multiple comparison gave a value of P<0.001 for the ADDL fyn^{+/+} data compared to all other conditions.

These results confirm that loss of Fyn kinase protected DG and CA3 hippocampal regions from cell death induced by amyloid β peptide assemblies. The results validate that amyloid β peptide assembly toxicity is mediated by a mechanism blocked by knockout of Fyn protein tyrosine kinase. These results further suggest that neuroprotective benefits can be obtained by treatments that L abrogate the activity of Fyn protein tyrosine kinase or the expression of the gene encoding Fyn protein kinase.

### EXAMPLE 16

### Astrocyte Activation Experiments

To investigate further the potential involvement of signal transduction in amyloid β peptide assembly toxicity, the experiments in this example compared the impact of amyloid β peptide assemblies on activation of astrocytes. For these experiments, cortical astrocyte cultures were prepared from neonatal (1-2 day old) Sprague-Dawley rat pups according to established methods (Levison, et al. (1991) In: Banker et al. (Eds.), Culturing Nerve Cells, MIT press, Cambridge, Mass., 309-36; Hu, et al. (1996) J. Biol. Chem. 271:2543-2547). Briefly, cerebral cortex was dissected out, trypsinized, and cells were cultured in α-MEM (GIBCO-BRL) containing 10% fetal bovine serum (Hyclone Laboratories Inc., Logan, Utah) and antibiotics (100 U/mL penicillin, 100 mg/mL streptomycin). After 11 days in culture, cells were trypsinized and replated into 100-mm plates at a density of ˜6×10⁵ cells/plate and grown until confluent (Hu, et al. (1996) supra).

Astrocytes were treated with amyloid β peptide assemblies prepared according to Example 1, or with amyloid β 17-42 (synthesized as per Lambert, et al. (1994) J. Neurosci. Res. 39:377-384; also commercially available). Treatment was carried out by trypsinizing confluent cultures of astrocytes and plating them onto 60-mm tissue culture dishes at a density of 1×10⁶ cells/dish (e.g., for RNA analysis and ELISAs), into 4-well chamber slides at 5×10⁴ cells/well (e.g., for immunohistochemistry), or into 96-well plates at a density of 5×10⁴ cells/well (e.g., for NO assays). After 24 hours of incubation, the cells were washed twice with PBS to remove serum, and the cultures incubated in α-MEM containing N2 supplements for an additional 24 hours before addition of amyloid β peptide or control buffer (i.e., buffer containing diluent).

Examination of astrocyte morphology was performed by examining cells under a NIKON TMS inverted microscope equipped with a JAVELIN SMARTCAM camera, SONY video monitor and color video printer. Typically, four arbitrarily selected microscopic fields (20× magnification) were photographed for each experimental condition. Morphological activation was quantified from the photographs with NIH Image by counting the number of activated cells (defined as a cell with one or more processes at least one cell body in length) in the four fields.

The mRNA levels in the cultures were determined with use of northern blot and slot blot analyses. This was carried out by exposing cells to amyloid β peptide assemblies or control buffer for 24 hours. After this time, the cells were washed twice with diethylpyrocarbonate (DEPC)-treated PBS, and total RNA was isolated by RNEASY purification mini-columns (QIAGEN, Inc., Chatsworth, Calif.), as recommended by the manufacturer. Typical yields of RNA were 8 to 30 mg of total RNA per dish. For northern blot analysis, 5 mg total RNA per sample was separated on an agarose-formaldehyde gel, transferred by capillary action to HYBOND-N membrane (Amersham, Arlington Heights Ill.), and UV crosslinked. For slot blot analysis, 200 ng of total RNA per sample was blotted onto DURALON-UV membrane (STRATAGENE, La Jolla Calif.) under vacuum, and UV crosslinked. Confirmation of equivalent RNA loadings was done by ethidium bromide staining or by hybridization and normalization with a GAPDH probe.

Probes were generated by restriction enzyme digests of plasmids, and subsequent gel purification of the appropriate fragment. Namely, cDNA fragments were prepared by RT-PCR using total RNA from rat cortical astrocytes. RNA was reverse transcribed with a SUPERSCRIPT II system (GIBCO-BRL), and PCR was performed on a PTC-100 thermal controller (MJ Research Inc, Watertown, Mass.) using 35 cycles at the following settings: 52° C. for 40 seconds; 72° C. for 40 seconds; 96° for 40 seconds. Primer pairs used to amplify a 447-bp fragment of rat IL-1β were: forward, 5′-GCA CCT TCT TTC CCT TCA TC-3′ (SEQ ID NO:66); and reverse, 5′-TGC TGA TGT ACC AGT TGG GG-3′ (SEQ ID NO:67). Primer pairs used to amplify a 435-bp fragment of rat GFAP were: forward, 5′-CAG TCC TTG ACC TGC GAC C-3′ (SEQ ID NO:68); and reverse, 5′ GCC TCA CAT CAC ATC CTT G-3′ (SEQ ID NO:69). PCR products were cloned into the pCR2.1 vector with the INVITROGEN TA cloning kit, and constructs were verified by DNA sequencing. Probes were prepared by EcoRI digestion of the vector, followed by gel purification of the appropriate fragments. The plasmids were the rat iNOS cDNA plasmid pAstNOS-4, corresponding to the rat iNOS cDNA bases 3007-3943 (Galea, et al. (1994) J. Neurosci. Res. 37:406-414), and the rat GAPDH cDNA plasmid pTRI-GAPDH (AMBION, Inc., Austin Tex.).

The probes (25 ng) were labeled with ³²P-dCTP by using a PRIME-A-GENE Random-Prime labeling kit (PROMEGA, Madison, Wis.) and separated from unincorporated nucleotides by use of push-columns (STRATAGENE). Hybridization was done under stringent conditions with QUIKHYB solution (STRATAGENE), using the protocol recommended for stringent hybridization. Briefly, prehybridization was conducted at 68° C. for about 30 to 60 minutes, and hybridization was at 68° C. for about 60 minutes. Blots were then washed under stringent conditions and exposed to either autoradiography or phosphoimaging plate. Autoradiograms were scanned with a BIO-RAD GS-670 laser scanner, and band density was L quantified with Molecular Analyst v2.1 (BIO-RAD, Hercules, Calif.) image analysis software. Phosphoimages were captured on a STORM 840 system (Molecular Dynamics, Sunnyvale Calif.), and band density was quantified with IMAGE QUANT v1.1 (Molecular Dynamics) image analysis software.

For measurement of NO by nitrite assay, cells were incubated with amyloid β peptides or control buffer for 48 hours, and then nitrite levels in the conditioned media were measured by the Griess reaction according to established methods (Hu, et al. (1996) supra). When the NOS inhibitor N-nitro-L-arginine methylester (L-name) or the inactive D-name isomer were used, these agents were added to the cultures at the same time as the amyloid β.

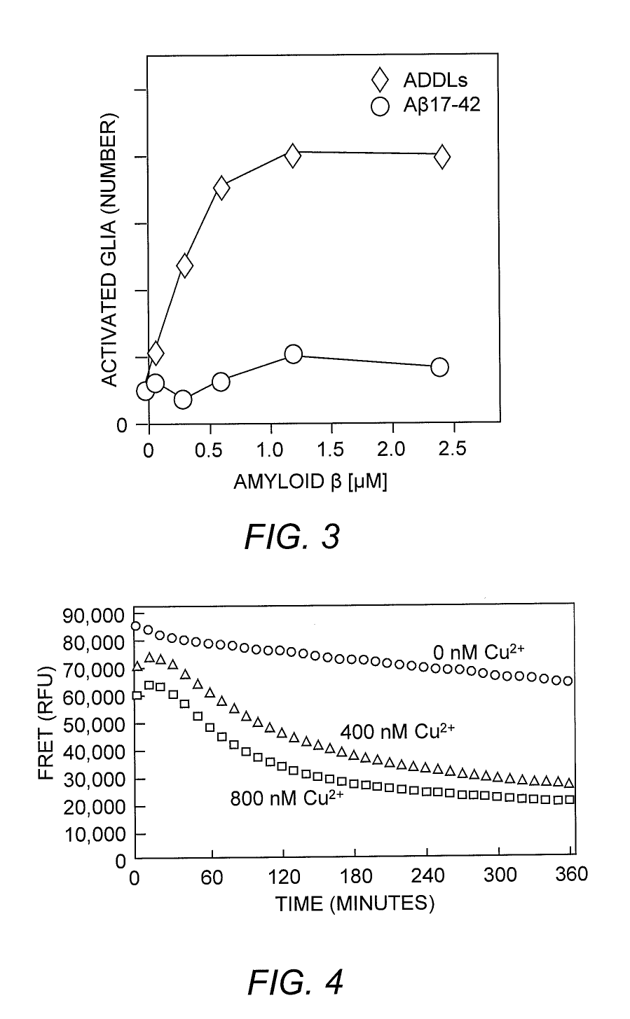

Results of these experiments are presented in FIG. 3. As can be seen in this figure, glia activation increased when astrocytes were incubated with amyloid β peptide assemblies, but not when astrocytes were incubated with amyloid β 17-42 peptide.

These results confirm that amyloid y peptide assemblies activate glial cells. It is possible that glial proteins may contribute to neural deficits, for instance, as occur in Alzheimer's Disease, and that some effects of amyloid β peptide assemblies may actually be mediated indirectly by activation of glial cells. Moreover, these results indicate that neuroprotective benefits can be obtained by treatments that modulate (i.e., increase or decrease) ADDL-mediated glial cell activation. Further, the results indicate that blocking these effects on glial cells, apart from blocking the neuronal effects, may be beneficial.

### EXAMPLE 17

### LTP Assay-ADDLs Disrupt LTP

Long-term potentiation (LTP) is a classic paradigm for synaptic plasticity and a model for memory and learning, faculties that are selectively lost in early stage AD. This example sets forth experiments performed to examine the effects of amyloid β peptide assemblies on LTP, particularly medial perforant path-granule cell LTP.

Injections of Intact Animals. Mice were anesthetized with urethane and placed in a sterotaxic apparatus. Body temperature was maintained using a heated water jacket pad. The brain surface was exposed through holes in the skull. Bregma and lambda positions for injection into the middle molecular layer of hippocampus are 2 mm posterior to bregma, 1 mm lateral to the midline, and 1.2-1.5 mm ventral to the brain surface. Amyloid β peptide assembly injections were by nitrogen puff through ˜10 nm diameter glass pipettes. Volumes of 20-50 nL of amyloid β peptide assembly solution (180 nM of amyloid β in PBS) were given over the course of an hour. Control mice received an equivalent volume of PBS alone. The animal was allowed to rest for varying time periods before the LTP stimulus is given (typically 60 minutes).

LTP in Injected Animals. Experiments follow the paradigm established by Routtenberg and colleagues for LTP in mice (Namgung, et al. (1995) Brain Research 689:85-92). Perforant path stimulation from the entorhinal cortex was used, with recording from the middle molecular layer and the cell body of the dentate gyrus. A population excitatory post-synaptic potential (pop-EPSP) and a population spike potential (pop-spike) were observed upon electrical stimulation. LTP could be induced in these responses by a stimulus of three trains of 400 Hz, 8×0.4 ms pulses/train (Namgung, et al. (1995) supra). Recordings were taken for 2-3 hours after the stimulus (i.e., applied at time 0) to determine if LTP was retained. The animal was then sacrificed immediately, or was allowed to recover for either 1, 3, or 7 days and then sacrificed as above. The brain was cryoprotected with 30% sucrose, and then sectioned (30 μM) with a microtome. Some sections were placed on slides subbed with gelatin and others were analyzed using a free-floating protocol. Immunohistochemistry was used to monitor changes in GAP-43, in PKC subtypes, and in protein phosphorylation of tau (PHF-1), paxillin, and focal adhesion kinase. Wave forms were analyzed by machine according to known methods (Colley, et al. (1990) J. Neurosci. 10:3353-3360). A 2-way ANOVA compared changes in spike amplitude between treated and untreated groups.

The results of this analysis indicated that amyloid β peptide assemblies blocked the persistence phase of LTP induced by high frequency electrical stimuli applied to entorhinal cortex and measured as cell body spike amplitude in middle molecular layer of the dentate gyrus.

After the LTP experiment was performed, animals were allowed to recover for various times and then sacrificed using sodium pentobarbitol anesthetic and perfusion with 4% paraformaldehye. For viability studies, times of 3 hours, 24 hours, 3 days, and 7 days were used. The brain was cryoprotected with 30% sucrose and then sectioned (30 μM) with a microtome. Sections were placed on slides subbed with gelatin and stained initially with cresyl violet. Cell loss was measured by counting cell bodies in the dentate gyrus, CA3, CA1, and entorhinal cortex, and correlated with dose and time of exposure of amyloid β peptide assemblies. The results of these experiments confirmed that no cell death occurred as of 24 hours following the LTP experiments.

Similarly, the LTP response was examined in hippocampal slices from young adult rats. The results of this analysis indicated that incubation of rat hippocampal slices with amyloid β peptide assemblies prevented LTP well before any overt signs of cell degeneration. Hippocampal slices (n=6) exposed to 500 nM amyloid β peptide assemblies for 45 minutes prior showed no potentiation in the population spike 30 minutes after the tetanic stimulation (mean amplitude 99%±7.6), despite a continuing capacity for action potentials. In contrast, LTP was readily induced in slices incubated with vehicle (n=6), with an amplitude of 138%±8.1 for the last 10 minutes; this value is comparable to that previously demonstrated in this age group (Trommer, et al. (1995) Exper. Neurol. 131:83-92). Although LTP was absent in ADDL-treated slices, their cells were competent to generate action potentials and showed no signs of degeneration.

These results validate that in both whole animals and tissue slices, the addition of amyloid β peptide assemblies results in significant disruption of LTP in less than an hour, prior to any cell degeneration or killing. These experiments thus indicate that amyloid β peptide assemblies exert very early effects, and interference with amyloid β peptide assembly formation and/or activity thus can be employed to obtain a therapeutic effect prior to advancement of a disease, disorder, or condition (e.g., Alzheimer's disease) to a stage where cell death results. In other words, these results confirm that decreases in memory occur before neurons die. Interference prior to such cell death thus can be employed to reverse the progression, and potentially restore decreases in memory.

### EXAMPLE 18

### Metal Complexes of Amyloid β Peptide Assemblies

The present invention also relates to the interaction of soluble, oligomeric assemblies of amyloid β peptide with metals. The interaction of amyloid β with metals has been suggested. See, e.g., U.S. Pat. No. 6,365,141; U.S. Application No. 20040013680; Liu, et al. (2006) J. Struct. Biol. 155(1):45-51; Miller, et al. (2006) J. Struct. Biol. 155(1):30-7; Stellato, et al. (2006) Eur. Biophys. J. 35(4):340-51; Syme & Viles (2006) Biochim. Biophys. Acta 1764(2):246-56; Zirah, et al. (2006) J. Biol. Chem. 281(4):2151-61; Karr, et al. (2005) Biochemistry, 44(14):5478-87; Maynard, et al. (2005) Int. J. Exp. Path. 86(3):147-59; Mekmouche, et al. (2005) Chembiochem. 6(9):1663-71; Raman, et al. (2005) J. Biol. Chem. 280(16):16157-62; Tickler, et al. (2005) J. Biol. Chem. 280(14):13355-63; Bishop & Robinson (2004) Brain Pathol. 14(4):448-52; Ciccotosto, et al. (2004) J. Biol. Chem. 279(41):42528-34; Huang, et al. (2004) J. Biol. Inorg. Chem. 9(8):954-60; Abramov, et al. (2003) J. Neurosci. 23(12):5088-95; Barnham, et al. (2003) J. Biol. Chem. 278(44):42959-65; Bush, et al. (2003) Proc. Natl. Acad. Sci. USA 100(20):11193-4; Bush (2003) Trends Neurosci. 26(4):207-14; Curtain, et al. (2003) J. Biol. Chem. 278(5):2977-82; Klug, et al. (2003) Eur. J. Biochem. 270(21):4282-93; Ritchie, et al. (2003) Arch. Neurol. 60(12):1685-91; Matsunaga, et al. (2002) Biochem. J. 361(Pt.3):547-56; Maynard, et al. (2002) J. Biol. Chem. 277(47):44670-6; Parbhu, et al. (2002) Peptides 23(7):1265-70; Zou, et al. (2002) J. Neurosci. 22(12):4833-4841; Curtain, et al. (2001) J. Biol. Chem. 276(23):20466-73; Yoshiike, et al. (2001) J. Biol. Chem. 276(34):32293-99; Atwood, et al. (2000) J. Neurochem. 75(3):1219-33; Miura, et al. (2000) Biochemistry 39(23):7024-31; Cherny, et al. (1999) J. Biol. Chem. 274(33):23223-8; Huang, et al. (1999) J. Biol. Chem. 274(52):37111-6; Clements, et al. (1996) J. Neurochem. 66(2):740-7. However, this example demonstrates that metal ions modulate assembly distribution and stabilize preexisting oligomers which are biologically active and neurotoxic.

HFIP-treated biotin-amyloid β 1-42 [Bpa10] film was dissolved in DMSO to 10 mM. Unbuffered phosphate or PBS, pH 7.4, with or without CuCl₂ at equimolar amounts relative to amyloid β peptide concentration, was added to bring peptide concentration to 1 mM and 100 μM for a total of eight different conditions:
1) 1 mM peptide in PBS+copper
2) 1 mM peptide in PBS no copper
3) 1 mM peptide in phosphate+copper
4) 1 mM peptide in phosphate no copper
5) 100 μM peptide in PBS+copper
6) 100 μM peptide in PBS no copper
7) 100 μM peptide in phosphate+copper
8) 100 μM peptide in phosphate no copper

All preparations were then incubated at 37° C. for 1 hour. Following incubation, the samples were centrifuged for 5 minutes at 13,000 RPM, split into two plates, and either cross-linked under UV light for one hour at room temperature or incubated in the dark for one hour at room temperature. LDS sample running buffer was subsequently added to each sample and the samples were separated on 4-12% Bis-Tris SDS-PAGE. Bands were visualized by silver stain.

SDS-PAGE analysis indicated that copper, for example, stabilized and enriched gel-stable oligomer species. In particular, stoichiometric CuCl₂ concentrations, relative to amyloid β 1-42 monomer, greatly stabilized the 12-mer band seen in non-copper-treated samples. The monomer band, in both the cross-linked and uncross-linked PBS samples+copper, nearly completely converted to 12-mer species, with a small amount converting into higher integral assemblies of 12-mers, such as 24-mers, 36-mers and 48-mers. A small amount of peptide was also retained at a trimer molecular weight.

Comparing the gel migration of uncross-linked native amyloid β 1-42 with that of the photo-crosslinked biotin-amyloid β 1-42 [Bpa10] peptide analog, cross-linking did not alter the Cu-induced stabilization of 12-mers. Importantly, copper addition and cross-linking did not change the migration of amyloid β 1-42 peptide species, but rather altered the assembly distribution and stabilize preexisting oligomers which are biologically active and neurotoxic.

In addition to SDS-PAGE analysis, a continuous fluorescence-based assay was used to analyze amyloid β assembly into oligomeric assemblies. This plate-based assembly assay allows for much higher throughput analysis of metals and solution conditions than the SDS-PAGE analyses. The fluorescence-based assay, a FRET-FP assay, was performed in a 384-well Corning® Non-Binding Surface black, opaque microtiter plate, and the assay mixtures were composed of the different buffers, metals and salts listed in Tables 6 and 7. These conditions were tested for their ability to stimulate oligomerization of various amyloid β peptide combinations. In particular, assay conditions listed in Table 6 were identified as conditions which accelerated and provided uniform amyloid β oligomerization of wild-type amyloid β 1-42 alone.

TABLE 6

| | Metal | [Buffer] | Buffer | | [peptide] | | [NaCl] |
|---|---|---|---|---|---|---|---|
| Assay | salt | (mM) | composition | pH | (nM) | Ratio* | (mM) |
| A | CuCl₂ | 25 | NaHCO₃/NaH₂PO₄ | 7.2 | 10 | 1:4 | 150 |
| B | FeCl₂ | 5 | HEPES/NaOH | 7.0 | 10 | 1:4 | |
| C | FeCl₃ | 5 | HEPES/NaOH | 7.0 | 10 | 1:4 | |
| D | MnCl₂ | 5 | HEPES/NaOH | 7.0 | 10 | 1:4 | |
| E | ZnCl₂ | 10 | HEPES/NaOH | 7.0 | 1000 | 5:0 | |
| F | MgCl₂ | 50 | MOPS/Tris | 8.05 | 1000 | 1:4 | |
| G | NiCl₂ | 25 | NaHCO₃/MOPS | 7.2 | 10 | 1:4 | 150 |
| H | CoCl₂ | 25 | NaHCO₃/MOPS | 7.2 | 10 | 1:4 | 150 |
| I | CrCl₃ | 25 | borax/MOPS | 7.2 | 10 | 1:4 | 150 |

*Ratio indicates FITC:wild-type peptide ratio.
HEPES, N-(2-hydroxyethyl)-piperazine-N′-2-ethanesulfonic acid.
MOPS, 3-Morpholinopropanesulfonic acid.
Tris, 2-amino-2-(hydroxymethyl)propane-1,3-diol.
Borax, Sodium tetraborate.

The assay conditions listed in Table 7 were used to demonstrate assembly formation using homogenous populations of amyloid β and mixtures of different amyloid β peptides.

TABLE 7

| | | Buffer | | | |
|---|---|---|---|---|---|
| | Metal | Composition | Aβ | FITC:native | |
| Assay | salt | (25 mM, pH 7.2) | species | peptide ratio | [NaCl] (mM) |
| J | CuCl₂ | NaHCO₃/NaH₂PO₄ | 10 nM Aβ 1-40 | 1:4 | 150 |
| K | CuCl₂ | NaHCO₃/NaH₂PO₄ | 2 nM Aβ 1-40 | FITC | 150 |
| K′ | CuCl₂ | NaHCO₃/NaH₂PO₄ | 8 nM Aβ 1-42 | native | 150 |
| L | CuCl₂ | NaHCO₃/NaH₂PO₄ | 2 nM Aβ 1-42 | FITC | 150 |
| L′ | CuCl₂ | NaHCO₃/NaH₂PO₄ | 8 nM Aβ 1-40 | native | 150 |
| M | CuCl₂ | NaHCO₃/NaH₂PO₄ | 10 nM DproNL | 1:4 | 150 |

Note,
amyloid β 1-40 did not significantly oligomerize in MgCl₂ assay condition, but did show assembly in other conditions.

All the peptides were dissolved in HFIP and dried to films. The films were resuspended in DMSO, and native and fluorescent peptides were mixed at this stage in the ratios indicated in Tables 6 and 7. These peptide mixtures were then diluted into 1×PBS (pH 7.4) according to the following to create working stocks: peptides assayed at 1 μM final concentration peptides were diluted to 100 μM at a final concentration of 20% DMSO; and peptides assayed at 10 nM final concentration were further diluted from this 100 μM stock to 1 μM in 20% DMSO in 1×PBS (pH 7.4). Amyloid β assembly was monitored on a TECAN GENIOS Pro plate reader, exciting at a wavelength of 485 nm and detecting emission at a wavelength of 535 nm. Kinetic traces were collected by recording fluorescence intensity and polarization readings every five minutes over a six-hour time course.

The results of this analysis demonstrated that copper could accelerate assembly of amyloid β 1-42 (see FIG. 4), and likewise, many other metals such as Mg, Zn, Fe, Co, Mn, Cr and Ni could also stimulate amyloid β 1-42 association (assays A-I, Table 6). In addition, Cu was observed to stimulate oligomerization of the amyloid β 1-40 peptide (assay J, Table 7), as well as of the amyloid β 1-42 [Nle35-DPro37] peptide (assay M, Table 7). However, amyloid β 1-40 did not oligomerize in the presence of Mg, while amyloid β 1-42 did, indicating potential metal selectivity for different association events. Amyloid β 1-42 [Nle35-Dpro37], a peptide which remains at LMW under most conditions, including Cu incubation appeared to arrest assembly at trimer. In addition to these homomeric association events, amyloid β 1-42 and amyloid β 1-40 were able to co-associate in the presence of Cu (assays K-L, Table 7), but not in the presence of Mg.

Another assay used to analyze amyloid β peptide assembly is size exclusion chromatography (SEC). To analyze the Cu and cross-linking conditions where Cu appeared to stabilize the 12-mer species relative to others, samples were prepared under identical conditions. After HFIP dissolution and dry film preparation, peptides were dissolved in anhydrous DMSO at 10 mM final concentration. A solution of 1×PBS (pH 7.4) buffer supplemented with 100 μM CuCl₂ was then added to dilute the peptides to 100 μM total concentration. Amyloid β peptide assembly occurred overnight at 37° C., and the cross-linked sample was irradiated for 1 hour while the non-crosslinked amyloid β peptide assemblies were left under ambient light. Samples were centrifuged at 13,000 rpm for 5 minutes, then loaded onto a SUPERDEX-200 HR 10/30 column, which is effective at separating proteins and oligomers lower than 500 kD molecular weight (MW), for size-exclusion chromatography separation. Protein elution was detected by absorbance at 220 nm.

When the Bpa-crosslinked peptide was compared to the native, Cu-incubated amyloid β 1-42 species, the results indicated that strong cross-linking was induced in the biotin-amyloid β 1-42 [Bpa10] peptide. The elution of uncross-linked native amyloid β 1-42 showed the lack of HMW material formation. The data indicated that oligomerization arrested at elution positions corresponding to MW values which were correlated to between 50 and 100 kD. While the SDS-PAGE migration of these two preps was similar, SEC analysis clearly demonstrated that the cross-linking induces larger assemblies due to inter-oligomer covalent attachment. The broad 50 kD to 100 kD peak of the Cu-amyloid β 1-42 sample, indicated that metal binding may be enriching for species in the 12-mer to 24-mer size range.

Because precipitate was observed prior to SEC column loading, the detergent SDS was used to make oligomers more soluble. HFIP-treated and dried amyloid β 1-42 peptide was dissolved in anhydrous DMSO at 10 mM final concentration. A solution of 1×PBS (pH 7.4) containing 100 μM CuCl₂±0.04% SDS was added to adjust the final peptide concentration to 100 μM, and the samples were incubated overnight at 37° C. Samples were centrifuged at 13,000 rpm for 5 minutes and loaded onto a SUPERDEX-200 HR 10/30 column for SEC separation. Protein elution was detected by absorbance at 220 nm. The addition of SDS to the samples was found to increase absorbance of the treated samples. This indicates better recovery and solubility of peptide oligomers. The elution profile was, however, very similar to non-SDS-treated samples, with no HMW material present in the chromatogram. Therefore, SDS enhances protein recover of Cu-induced amyloid β peptide assembly formation with minimal perturbation of SEC elution.

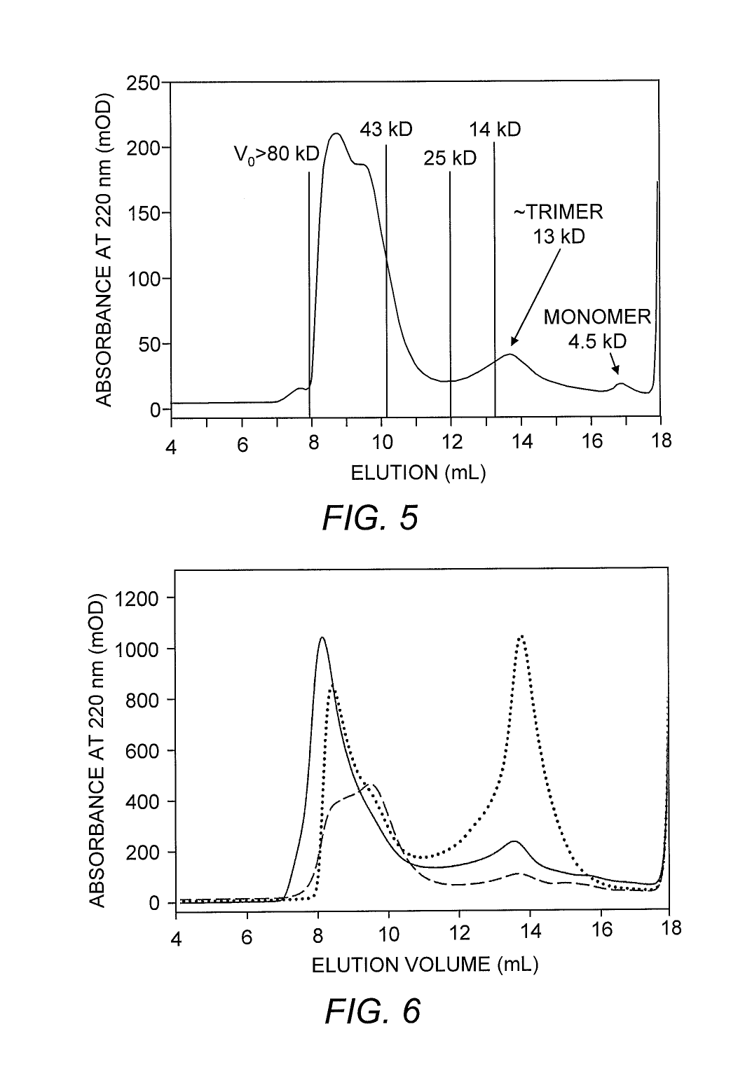

Because of the enhanced solubility and defined MW range of oligomerization, the Cu-SDS solution was used for further optimize the amyloid β peptide assembly preparative method. HFIP-treated and dried amyloid β 1-42 peptide was dissolved in anhydrous DMSO at 10 mM final concentration. A solution of 1×PBS (pH 7.4) containing 100 μM CuCl₂±0.04% SDS was added to adjust the final peptide concentration to 100 μM, and the sample was incubated overnight at 37° C. The sample was centrifuged at 13,000 rpm for 5 minutes and loaded onto a SUPERDEX-75 HR 10/30 column for SEC separation. Protein elution was detected by absorbance at 220 nm. This analysis indicated that intermediate MW (IMW) oligomer species between 43 and 80 kD and low MW species, presumably trimeric, were enhanced (FIG. 5). In addition, the IMW oligomers appeared to elute with higher relative yield from this column than from the SUPERDEX-200 column. An observed oligomer doublet may have been due to distinct structural forms of the same subunit composition, such as 12-mer, which had different degrees of compactness and hence different hydrodynamic radii. Alternatively, the doublet peak could have been due to the association of one or more LMW species with a 12-mer IMW oligomer.

To determine whether the various amyloid β peptides could yield these IMW oligomer species, oligomerization of amyloid β 1-42, amyloid β 1-43, and amyloid β 1-40 was analyzed. HFIP-treated and dried peptides were dissolved in anhydrous DMSO at 10 mM final concentration. A solution of 1×PBS (pH 7.4) containing 100 μM CuCl₂ and 0.04% SDS was added to adjust the final peptide concentration to 250 μM, and the samples were incubated 1 hour at room temperature. Samples were centrifuged at 13,000 rpm for 5 minutes and loaded onto a SUPERDEX-75 HR 10/30 column for SEC separation. Protein elution was detected by absorbance at 220 nm. This analysis indicated that amyloid β 1-42 was most efficient at forming IMW oligomers, with amyloid β 1-43 being somewhat less prone to IMW assembly, and amyloid β 1-40 exhibiting less IMW oligomer propensity (FIG. 6).

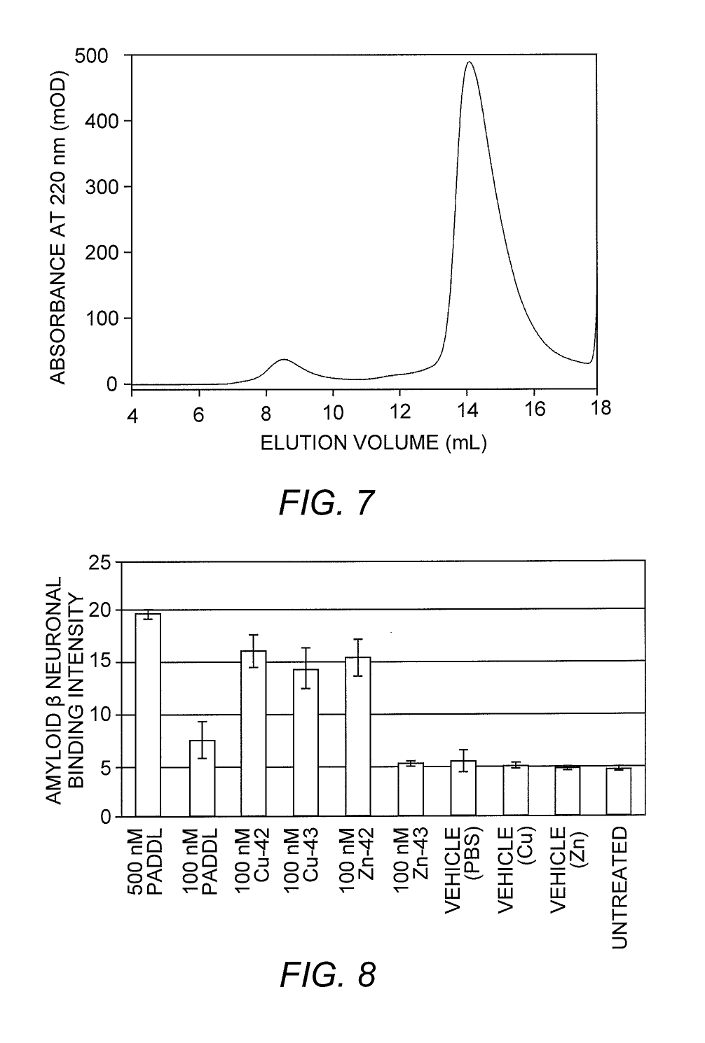

To further evaluate this modified preparative method, the amyloid β 1-42[Nle35-DPro37] peptide analog was used to prepare amyloid β peptide assemblies. HFIP-treated and dried amyloid β 1-42 [Nle35-DPro37] peptide was dissolved in anhydrous DMSO at 10 mM final concentration. A solution of 1×PBS (pH 7.4) containing 100 μM CuCl₂ and 0.04% SDS was added to adjust the final peptide concentration to 100 μM, and the sample was incubated overnight at 37° C. Samples were centrifuged at 13,000×g for 5 minutes and loaded onto a SUPERDEX-75 HR 10/30 column for SEC separation. Protein elution was detected by absorbance at 220 nm. Peptide assemblies eluted at a position roughly corresponding to the trimers observed using amyloid β 1-42, amyloid β 1-43, and amyloid β 1-40 peptides, with very low levels of IMW and/or void volume material (FIG. 7). This analysis showed the extremely low conversion of the amyloid β 1-42 [Nle35-DPro37] peptide analog into IMW oligomers, likely due to the stabilized beta-hairpin near the peptide's C-terminus.

As a final step in preparative method development, it was determined whether amyloid β 1-42 peptide assemblies formed in the presence of Cu and SDS could be frozen and thawed. In this regard, a post-void volume SUPERDEX-75 oligomer fraction containing Cu-Biotin-amyloid β 1-42 peptide assemblies was snap frozen in liquid N₂ after 50% glycerol was added to a final concentration of 10% as a cryoprotectant. The results of this analysis indicated that there was very little loss in oligomeric IMW stable species. Thus, this method and composition will allow for long term storage of IMW ADDLs and their repeated use in biochemical and functional assays.

To determine whether metal complexes of amyloid β peptide assemblies may have a role in vivo, binding assays with metal complexes were conducted. Primary hippocampal neurons were incubated for 10 minutes at 37° C. with equal concentrations of oligomerized amyloid β 1-42, amyloid β 1-43, and amyloid β 1-40 and respective vehicle controls. Subsequently, neurons were fixed for 10 minutes at 4° C. with 4% paraformaldehyde and washed three times with in phosphate-buffered saline. Non-specific binding was blocked using 5% BSA in phosphate-buffered saline for 1 hour and neurons were subsequently incubated with the oligomer-selective antibody 20C2 overnight. Neurons were washed three times with phosphate-buffered saline and incubated with CY5 labeled secondary anti-mouse antibody for two hours at room temperature, washed three times with phosphate-buffered saline and imaged using the ARRAYSCAN imaging platform. Alternatively, detection of biotinylated oligomer species of amyloid β 1-42, and amyloid β 1-40 was employed using SA-TRITC labeling.

Images of labeled neurons were acquired and analyzed on a CELLOMICS ARRAYSCAN HCS Reader. Acquisition settings included imaging 10 fields per well at a 10× magnification. A proprietary modification of a CELLOMICS BioApplication was used for image analysis. Nuclei were identified using DAPI (Channel 1) and neurons were identified and selected for analysis by their staining by the MAP2 antibody (Channel2). The neuronal subpopulation was analyzed for amyloid β binding in Channel 3. The BioApplication automatically reports the level of amyloid β peptide assembly binding in each individual cell. Images and numeric data were automatically transferred to CELLOMICS Store, where well- and cell-level data were viewed for analysis. Binding Intensity values for all preparations were exported to EXCEL.

Copper and zinc containing preparations of amyloid β 1-42 and amyloid β 1-43 peptide assemblies showed intense labeling of dendritic spines in primary hippocampal neurons. Compared to equal concentrations of a standard amyloid β peptide assembly preparation, the metal-containing amyloid β 1-42 peptide assemblies showed a 2.5-fold increase in binding intensity (FIG. 8). Spine-specific, although lower intensity, staining was observed in assemblies containing amyloid β 1-43.

Biotinylated oligomer assemblies showed equivalent dendritic binding behavior to native amyloid β peptide assemblies and could be equally well detected with oligomer-selective antibodies as well as streptavidin conjugates. Low molecular weight species did not display visible dendritic binding and corresponded to vehicle treated controls in binding intensity.

### EXAMPLE 19

### Early Effects of ADDLs In Vivo

The primary symptoms of Alzheimer's disease involve learning and memory deficits. However, the link between behavioral deficits and aggregated amyloid deposits has been difficult to establish. In transgenic mice, overexpressing mutant APP under the control of the platelet-derived growth factor promoter results in the deposition of large amounts of amyloid (Games, et al. (1995) Nature 373:523-527). By contrast, no behavioral deficits have been reported using this system. Other researchers (i.e., Nalbantoglu, et al. (1997) Nature 387:500-505; Holcomb, et al. (1998) Nat. Med. 4:97-100) working with transgenic mice report observing significant behavioral and cognitive deficits that occur well before any significant deposits of aggregated amyloid are observed. These behavioral and cognitive defects include failure to long-term potentiate (Nalbantoglu, et al. (1997) supra). It is now believed that these models collectively suggest that non-deposited forms of amyloid are responsible for the early cognitive and behavioral deficits that occur as a result of induced neuronal malfunction. It is consistent with these models that the novel amyloid β peptide assemblies described herein are this non-deposited form of amyloid causing the early cognitive and behavioral defects. In view of this, amyloid β peptide assembly modulating compounds can be employed in the treatment and/or prevention of these early cognitive and behavioural deficits resulting from ADDL-induced neuronal malfunction, or amyloid β peptide assemblies themselves can be applied, for instance, in animal models, to study such induced neuronal malfunction.

Similarly, in elderly humans, cognitive decline and focal memory deficits can occur well before a diagnosis of probable stage I Alzheimer's disease is made (Linn, et al. (1995) Arch. Neurol. 52:485-490). These focal memory deficits may result from induced aberrant signaling in neurons, rather than cell death. Other functions, such as higher order writing skills (Snowdon, et al. (1996) JAMA, 275:528-532) also may be affected by aberrant neuronal function that occurs long before cell death. It is consistent with what is known regarding these defects, and the information regarding amyloid β peptide assemblies provided herein, that amyloid β peptide assemblies induce these defects in a manner similar to compromised LTP function such as is induced by amyloid β peptide assemblies. Along these lines, amyloid β peptide assembly modulating compounds according to the invention can be employed in the treatment and/or prevention of these early cognitive decline and focal memory deficits, and impairment of higher order writing skills, resulting from amyloid β peptide assembly formation or activity, or amyloid β peptide assemblies themselves can be applied, for instance, in animal models, to study such induced defects. In particular, such studies can be conducted such as is known to those skilled in the art, for instance by comparing treated or placebo-treated age-matched subjects.

What is claimed is:

1. An isolated soluble amyloid β peptide assembly comprising at least three amyloid β peptide subunits, wherein at least one subunit is an amyloid β X-Y peptide analog,
wherein X is an integer between 1 and 28, and Y is 40, 42 or 43; and
wherein when X is 1, the amino acid residue at position 1 is an aspartic acid or a modified amino acid residue.

2. The assembly of claim 1, wherein the analog further comprises a label.

3. The assembly of claim 1, wherein the analog further comprises one or two additional carboxyl-terminal amino acid residues.

4. The assembly of claim 1, wherein the analog comprises the amino acid sequence R₂-Xaa₁-Gly-Ala-Ile-Ile-Gly-Leu-Xaa₂-Val-Xaa₃-Xaa₄-Val-Val-R₃ (SEQ ID NO:65),
wherein R₂ denotes a peptide of 0 to 29 amino acid residues in length; Xaa₁ is a basic amino acid residue; Xaa₂ is methionine or a structural derivative thereof; Xaa₃ and Xaa₄ are independently glycine, D-proline or L-proline, with the proviso that both Xaa₃ and Xaa₄ are not simultaneously a proline; and R₃ is a peptide of 0 to 5 amino acid residues in length.

5. The assembly of claim 1, further comprising one or more metal atoms.

6. The assembly of claim 1, wherein said assembly is produced from a homogenous population of amyloid β peptide subunits.

7. The assembly of claim 1, wherein said assembly is produced from a heterogenous population of amyloid β peptide subunits.
